(19) United States
(12) Patent Application Publication
(10) Pub. No.: US 2025/0360502 A1
(43) Pub. Date: Nov. 27, 2025

(54) MICROFLUIDIC DEVICE FOR ANALYZING STEADY STATE BIOLOGICAL REACTIONS

(71) Applicant: Yeda Research and Development Co. Ltd., Rehovot (IL)

(72) Inventors: Roy BAR-ZIV, Rehovot (IL); Shirley SHULMAN DAUBE, Rehovot (IL); Eyal KARZBRUN, Rehovot (IL); Alexandra TAYAR, Rehovot (IL); Joshua Emmanuel RICOUVIER, Rehovot (IL); Omer SHABTAI, Rehovot (IL); Pavel MOSTOV, Rehovot (IL)

(73) Assignee: Yeda Research and Development Co. Ltd., Rehovot (IL)

(21) Appl. No.: 18/872,303

(22) PCT Filed: Jun. 7, 2023
(86) PCT No.: PCT/IL2023/050590

§ 371 Date: Dec. 6, 2024

Related U.S. Application Data
(60) Provisional application No. 63/349,717, filed on Jun. 7, 2022.

(51) Int. Cl. B01L 3/00; C12P 21/00; G01N 33/543

(52) U.S. Cl. CPC B01L 3/502715; B01L 3/50273; B01L 3/502746; C12P 21/00; G01N 33/543; B01L 2200/027; B01L 2200/0694; B01L 2300/0816; B01L 2300/0861; B01L 2400/0472

(57) ABSTRACT

A microfluidic device is disclosed which comprises:
(i) a first surface which comprises a plurality of reaction units, each reaction unit having a test chamber connected to at least one opening via a feeding capillary, wherein said at least one opening is in communication with a hollow along a depth of the device, said hollow being defined by internal walls; and
(ii) a second surface which comprises at least one flow-through channel having at least one inlet port and at least one outlet port,
wherein said hollow extends to said second surface and fluidly connects said test chamber with said flow-through channel.

### RELATED APPLICATION/S

This application claims the benefit of priority of U.S. Provisional Patent Application No. 63/349,717, filed on 7 Jun. 2022, the contents of which are incorporated herein by reference in their entirety.

### FIELD AND BACKGROUND OF THE INVENTION

The present invention, in some embodiments thereof, relates to a microfluidic device for analyzing a plurality of biological/chemical reactions at steady-state.

Studying mechanisms responsible for information transfer and emergent collective behavior in a system of communicating cells such as a developing embryo, presents major experimental challenges, as many processes are inherently coupled and there is insufficient control over the multitude of parameters in a living system. The fundamental model of multicellular communications by diffusible signals is the reaction-diffusion equation of the signaling molecules, which can describe wide range of naturally found phenomena such as synchrony and pattern formation. Molecular signals generated in cells diffuse between neighboring cells, which in turn elicit cellular response encoded in gene regulatory networks (GRN). A reaction-diffusion regime is described by a differential equation of the local concentration of diffusible molecular signal p(\vec{x}, t) which changes in time according to diffusion in space and a local non-linear reaction: \dot{p}=DV²p+f(p). The non-linear term includes a degradation term that allows achieving steady state.

Cell-free protein synthesis (CFPS) systems have recently emerged as a technique to study principles of gene networks and inter-cellular communication in living systems using synthetic minimalistic and simplified model systems. CFPS systems also provide the means to develop synthetic programmable cell-free systems mimicking living systems, which would be capable of autonomous computation for applications of diagnostics, sensing, evolution, interfacing with living systems, etc.

The challenges in assembling artificial systems capable of multicellular interactions have been effective turnover at the cellular scale, control over the spatial distribution of the reactions, and achieving steady state cell-free gene expression conditions.

Background art includes U.S. Pat. Nos. 8,592,221, 8,449,837, International Patent Application WO2008/090557, International Patent Application No. WO2021/059269 and U.S. Pat. No. 11,396,012.

### SUMMARY OF THE INVENTION

According to an aspect of the present invention, there is provided a microfluidic device comprising:
(i) a first surface which comprises a plurality of reaction units, each reaction unit having a test chamber connected to at least one opening via a feeding capillary, wherein the at least one opening is in communication with a hollow along a depth of the device, the hollow being defined by internal walls; and
(ii) a second surface which comprises at least one flow-through channel having at least one inlet port and at least one outlet port,
wherein the hollow extends to the second surface and fluidly connects the test chamber with the flow-through channel.

According to another aspect of the present invention, there is provided a microfluidic device comprising:
(i) a first surface which comprises a plurality of reaction units, each reaction unit having a test chamber connected to at least one opening via a feeding capillary, wherein the at least one opening is in communication with a hollow along a depth of the device, the hollow being defined by internal walls, wherein a biomolecule is attached to a surface of at least a first portion of the test chamber, the biomolecule being a component of a cellular process, and wherein an immobilizing agent is optionally attached to a surface of at least a second portion of the test chamber, the immobilizing agent capable of immobilizing a product of the cellular process; and
(ii) a second surface which comprises at least one flow-through channel having at least one inlet port and at least one outlet port,
wherein the hollow extends to the second surface and fluidly connects the test chamber with the flow-through channel;
wherein the at least one flow-through channel, the feeding capillary and the at least one opening being of dimensions, so as to ensure the cellular process reaches and is maintained in a steady-state.

According to embodiments of the invention, the at least one flow-through channel, the feeding capillary and the at least one opening being of dimensions to allow diffusion of a molecule from the flow-through channel via the hollow to the test chamber of the reaction unit and vice-versa and reduce flow of fluid in the reaction unit.

According to embodiments of the invention, the at least one flow-through channel, the feeding capillary and the at least one opening being of dimensions to allow diffusion of a component of a cellular process from the flow-through channel to the test chamber of the reaction unit via the hollow and reduce flow of fluid in the reaction unit

According to embodiments of the invention, at least one the reaction unit further comprises at least one interconnecting capillary which connects a test chamber of a first reaction unity of the plurality of reaction units to a test chamber of a second reaction unit of the plurality of reaction units.

According to embodiments of the invention, at least a portion of the plurality of reaction units are organized in a grid such that at least one test chamber of a reaction unit of the plurality of reaction units is connected to four test chambers of corresponding reaction units via interconnecting capillaries.

According to embodiments of the invention, the depth ratio of the reaction unit: flow-through channel is greater than 1:5.

According to embodiments of the invention, the hydrodynamic resistance in the feeding capillary is at least 10³ times greater than the hydrodynamic resistance in the flow-through channel.

According to embodiments of the invention, the hydrodynamic resistance in the interconnecting capillary is at least 10³ times greater than the hydrodynamic resistance in the flow-through channel.

According to embodiments of the invention, the width of the interconnecting capillary is identical to the width of the feeding capillary.

According to embodiments of the invention, the width ratio of the feeding capillary: flow-through channel is greater than 1:3.

According to embodiments of the invention, the depth of the reaction unit is about 1 micron to about 50 microns.

According to embodiments of the invention, the depth of the flow-through channels is about 20 microns to about 250 microns.

According to embodiments of the invention, the diameter of the hollow is between 5-50 microns.

According to embodiments of the invention, the volume of the hollow is about 3-10 times a volume of the test chamber.

According to embodiments of the invention, the length of the feeding capillary of a first reaction unit of the plurality of reaction units is identical to a length of the feeding capillary of a second reaction unit of the plurality of reaction units.

According to embodiments of the invention, the length of the feeding capillary of a first reaction unit of the plurality of reaction units is non-identical to a length of the feeding capillary of a second reaction unit of the plurality of reaction units.

According to embodiments of the invention, the test chamber is between 20-100 microns in diameter.

According to embodiments of the invention, the device is sealed with a sealant.

According to embodiments of the invention, the sealant is gas permeable.

According to embodiments of the invention, a biomolecule is attached to a surface of at least a portion of the test chamber, the biomolecule being:
(i) a component of a cellular process; and/or
(ii) an immobilizing agent capable of immobilizing a product of the cellular process.

According to embodiments of the invention, the cellular process comprises protein expression.

According to embodiments of the invention, the component of a cellular process comprises a nucleic acid.

According to embodiments of the invention, aa sequence of the nucleic acid encodes a promoter operatively linked to a nucleic acid sequence encoding a polypeptide.

According to embodiments of the invention, the polypeptide is a detectable polypeptide.

According to embodiments of the invention, the promoter is a tissue-specific promoter.

According to embodiments of the invention, the polypeptide is a transcription factor.

According to embodiments of the invention, the microfluidic is of dimensions such that the polypeptide expressed from the nucleic acid forms a gradient in the feeding capillary.

According to embodiments of the invention, the sequence of the isolated nucleic acid in a test chamber of a first reaction unit of the plurality of reaction units is different to the sequence of the isolated nucleic acid in a test chamber of a second reaction unit of the plurality of reaction units.

According to embodiments of the invention, the promoter is a constitutive promoter.

According to embodiments of the invention, the promoter is a non-constitutive promoter.

According to embodiments of the invention, the nucleic acid is attached to the surface via a reactive group.

According to embodiments of the invention, the reactive group is photoreactivatable.

According to embodiments of the invention, the photoreactivatable reactive group is selected from the group consisting of amine, hydroxy, thiohydroxy, halo, alkoxy, thioalkoxy, aryloxy, thioaryloxy, carboxylate, phosphate, phosphonate, sulfate and sulfonate.

According to embodiments of the invention, the nucleic acid sequence comprises a plurality of nucleic acid sequences.

According to embodiments of the invention, the plurality of nucleic acid sequences encode a transcriptome.

According to embodiments of the invention, the immobilizing agent comprises an antibody.

According to embodiments of the invention, the product of the cellular process comprises a ribosome.

According to embodiments of the invention, the microfluidic device is fabricated from a substrate having attached thereto a plurality of monolayers the monolayers being composed of a compound which comprises a general formula I:

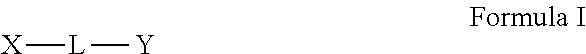

wherein:
X is a functionalized group capable of binding to the substrate;
L is a polymer capable of forming the monolayer onto the substrate; and
Y is a photoactivatable group capable of generating a reactive group upon exposure to the light.

According to another aspect of the present invention, there is provided a method of expressing a polypeptide comprising contacting the isolated nucleic acid of the microfluidic device of any one of claims 24-39 with a composition which comprises components for performing expression of the polypeptide from the isolated nucleic acid, under conditions that allow expression of the polypeptide, thereby expressing the polypeptide.

According to embodiments of the invention, the composition comprises a cell extract.

According to embodiments of the invention, the cell extract is devoid of nucleic acids.

According to embodiments of the invention, the protein forms a gradient in the reaction unit.

According to embodiments of the invention, the expressing is effected for at least 2 hours.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### General Terminology

As used herein, the term “amine” describes both a —NR′R″ group and a —NR′— group, wherein R′ and R″ are each independently hydrogen, alkyl, cycloalkyl, aryl, as these terms are defined herein.

The amine group can therefore be a primary amine, where both R′ and R″ are hydrogen, a secondary amine, where R′ is hydrogen and R″ is alkyl, cycloalkyl or aryl, or a tertiary amine, where each of R′ and R″ is independently alkyl, cycloalkyl or aryl.

Alternatively, R′ and R″ can each independently be hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, carbonyl, C-carboxylate, O-carboxylate, N-thiocarbamate, O-thiocarbamate, urea, thiourea, N-carbamate, O-carbamate, C-amide, N-amide, guanyl, guanidine and hydrazine.

The term “amine” is used herein to describe a —NR′R″ group in cases where the amine is an end group, as defined hereinunder, and is used herein to describe a —NR′— group in cases where the amine is a linking group.

Herein throughout, the phrase “end group” describes a group (a substituent) that is attached to another moiety in the compound via one atom thereof.

The phrase “linking group” describes a group (a substituent) that is attached to another moiety in the compound via two or more atoms thereof.

The term “alkyl” describes a saturated aliphatic hydrocarbon including straight chain and branched chain groups. Preferably, the alkyl group has 1 to 20 carbon atoms. Whenever a numerical range; e.g., “1-20”, is stated herein, it implies that the group, in this case the alkyl group, may contain 1 carbon atom, 2 carbon atoms, 3 carbon atoms, etc., up to and including 20 carbon atoms. More preferably, the alkyl is a medium size alkyl having 1 to 10 carbon atoms. Most preferably, unless otherwise indicated, the alkyl is a lower alkyl having 1 to 4 carbon atoms. The alkyl group may be substituted or unsubstituted. Substituted alkyl may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, N-carbamate, O-carbamate, C-amide, N-amide, guanyl, guanidine and hydrazine.

The alkyl group can be an end group, as this phrase is defined hereinabove, wherein it is attached to a single adjacent atom, or a linking group, as this phrase is defined hereinabove, connecting another moiety at each end thereof.

The term “cycloalkyl” describes an all-carbon monocyclic or fused ring (i.e., rings which share an adjacent pair of carbon atoms) group where one or more of the rings does not have a completely conjugated pi-electron system. The cycloalkyl group may be substituted or unsubstituted. Substituted cycloalkyl may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, N-carbamate, O-carbamate, C-amide, N-amide, guanyl, guanidine and hydrazine. The cycloalkyl group can be an end group, as this phrase is defined hereinabove, wherein it is attached to a single adjacent atom, or a linking group, as this phrase is defined hereinabove, connecting two or more moieties at two or more positions thereof.

The term “aryl” describes an all-carbon monocyclic or fused-ring polycyclic (i.e., rings which share adjacent pairs of carbon atoms) groups having a completely conjugated pi-electron system. The aryl group may be substituted or unsubstituted. Substituted aryl may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, N-carbamate, O-carbamate, C-amide, N-amide, guanyl, guanidine and hydrazine. The alkyl group can be an end group, as this term is defined hereinabove, wherein it is attached to a single adjacent atom, or a linking group, as this term is defined hereinabove, connecting two or more moieties at two or more positions thereof.

The term “halide” or “halo” describes fluorine, chlorine, bromine or iodine.

The term “haloalkyl” describes an alkyl group as defined above, further substituted by one or more halide.

The term “sulfate” describes a —O—S(═O)₂—OR′ end group, or an —O—S(═O)₂—O— linking group, as these phrases are defined hereinabove, where R′ is as defined hereinabove. This term further encompasses thiosulfates.

The term “thiosulfate” describes a —O—S(═S)(═O)—OR′ end group or a —O—S(═S)(═O)—O— linking group, as these phrases are defined hereinabove, where R′ is as defined hereinabove.

The term “sulfonate” describes a —S(═O)₂—R′ end group or an —S(═O)₂— linking group, as these phrases are defined hereinabove, where R′ is as defined herein.

The term “sulfonamide” describes a —S(═O)₂—NR′R″ end group or a —S(═O)₂—NR′— linking group, as these phrases are defined hereinabove, with R′ and R″ as defined herein. This term encompasses the terms N-sulfonamide and S-sulfonamide.

The term “N-sulfonamide” describes an R′S(═O)₂—NR″— end group or a —S(═O)₂—NR′— linking group, as these phrases are defined hereinabove, where R′ and R″ are as defined herein.

The term “S-sulfonamide” describes an —S(═O)₂—NR′R″— end group or a —S(═O)₂—NR′— linking group, as these phrases are defined hereinabove, where R′ and R″ are as defined herein.

The term “phosphonate” describes a —P(═O)(OR′)(OR″) end group or a —P(═O)(OR′)(O)— linking group, as these phrases are defined hereinabove, with R′ and R″ as defined herein.

The term “phosphate” describes an —O—P(═O)₂(OR′) end group or a —O—P(═O)₂(O)— linking group, as these phrases are defined hereinabove, with R′ as defined herein. This term further encompasses the term thiophosphonate.

The term “thiophosphate” describes an —O—P(═O)(═S)(OR′) end group or a —O—P(═O)(═S)(O)— linking group, as these phrases are defined hereinabove, with R′ as defined herein.

The term “carbonyl” or “carbonylate” as used herein, describes a —C(═O)—R′ end group or a —C(═O)— linking group, as these phrases are defined hereinabove, with R′ as defined herein.

Alternatively, R′ can be halide, or any other reactive derivative. This term encompasses the term “thiocarbonyl”.

The term “thiocarbonyl” as used herein, describes a —C(═S)—R′ end group or a —C(═S)— linking group, as these phrases are defined hereinabove, with R′ as defined herein.

The term “hydroxyl” describes a —OH group.

The term “alkoxy” describes both an —O-alkyl and an —O-cycloalkyl group, as defined herein.

The term “aryloxy” describes both an —O-aryl and an —O-heteroaryl group, as defined herein.

The term “thiohydroxy” describes a —SH group.

The term “thioalkoxy” describes both a —S-alkyl group, and a —S-cycloalkyl group, as defined herein.

The term “thioaryloxy” describes both a —S-aryl and a —S-heteroaryl group, as defined herein.

The term “cyano” describes a —C≡N group.

The term “isocyanate” describes an —N═C═O group.

The term “nitro” describes an —NO₂ group.

The term “azo” describes an —N═NR′ end group or an —N═N— linking group, as these phrases are defined hereinabove, with R′ as defined hereinabove.

The term “carboxylate” describes a —C(═O)—OR′ end group or a —C(═O)—O— linking group, as these phrases are defined hereinabove, where R′ is as defined herein. This term encompasses the terms O-carboxylate, C-thiocarboxylate, and O-thiocarboxylate, as well as various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters.

The term “carbamate” describes an R″OC(═O)—NR′— end group or a —OC(═O)—NR′— linking group, as these phrases are defined hereinabove, with R′ and R″ as defined herein. This term encompasses the terms O-carbamate, thiocarbamate and include various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters.

The term “amide” describes a —C(═O)—NR′R″ end group or a —C(═O)—NR′— linking group, as these phrases are defined hereinabove, where R′ and R″ are as defined herein. This term encompasses the term N-amide.

The term “N-amide” describes a R′C(═O)—NR″— end group or a R′C(═O)—N— linking group, as these phrases are defined hereinabove, where R′ and R″ are as defined herein.

The term “hydrazine” describes a —NR′—NR″R′″ end group or a —NR′—NR″— linking group, as these phrases are defined hereinabove, with R′, R″, and R′″ as defined herein.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furan, thiophene, imidazole, oxazole, thiazole, pyrazole, pyridine, pyrimidine, quinoline, isoquinoline and purine. The heteroaryl group may be substituted or unsubstituted. Substituted heteroaryl may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, O-carbamate, N-carbamate, C-amide, N-amide, guanyl, guanidine and hydrazine. The heteroaryl group can be an end group, as this phrase is defined hereinabove, where it is attached to a single adjacent atom, or a linking group, as this phrase is defined hereinabove, connecting two or more moieties at two or more positions thereof. Representative examples are pyridine, pyrrole, oxazole, indole, purine and the like.

The term “heteroalicyclic” describes a monocyclic or fused ring group having in the ring(s) one or more atoms such as nitrogen, oxygen and sulfur. The rings may also have one or more double bonds. However, the rings do not have a completely conjugated pi-electron system. The heteroalicyclic may be substituted or unsubstituted. Substituted heteroalicyclic may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, O-carbamate, N-carbamate, C-amide, N-amide, guanyl, guanidine and hydrazine. The heteroalicyclic group can be an end group, as this phrase is defined hereinabove, where it is attached to a single adjacent atom, or a linking group, as this phrase is defined hereinabove, connecting two or more moieties at two or more positions thereof. Representative examples are piperidine, piperazine, tetrahydrofurane, tetrahydropyrane, morpholino and the like.

The term “ester” describes a moiety containing a carboxylate group, as defined herein.

An “alkenyl” group describes an alkyl group which consists of at least two carbon atoms and at least one carbon-carbon double bond.

An “alkynyl” group describes an alkyl group which consists of at least two carbon atoms and at least one carbon-carbon triple bond.

A “dienophile” group describes a group which comprises at least two conjugated double-double boned.

### BRIEF DESCRIPTION OF THE SEVERAL VIEWS OF THE DRAWINGS

Some embodiments of the invention are herein described, by way of example only, with reference to the accompanying drawings and images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-E 3D-feeding configuration. (A) Schematic of the overall chip design. Inlet shows immobilized DNA strands and gene expression process taking place inside the compartment. Arrows indicate regions of material transfer by flow and by diffusion. (B) Schematic of the geometry of a single compartment, and the feeding method: vertical through-holes. (C) Optical 3D reconstruction of a single compartment. The depth on the compartment h_{c}≈2.5 μm (D) SEM image of the capillary, showing the deep through-hole connecting the compartment to the flow on the other face of the silicon wafer. The radius of the hole rₕ=15 μm (E) bright field image of an area on the compartment layer.

FIGS. 2A-D. flow layer design. (A) flow layer scheme. This structure is carved 150 μm deep into the silicon wafer, on the opposite face to the compartment layer. The two large squares are the inlet\outlet reservoirs, which allow connection to external tubing. (B) bright field image of a section in the flow layer. (C) electric analogue of the flow between the layers. (D) fluorescent beads of radius 1 μm flowing through the channels. Four particles in different channels are tracked for 5 seconds. Average speed v≈50 μm/sec

FIGS. 3A-C. electrical model of the capillary-hole configuration. (A) 3D visualization of the entire compartment-capillary-hole structure, along with the physical parameters. (B) electrical model of the physical structure in (A). The dependence of the electrical parameters on the physical parameter is summarized in Table 1. (C) protein concentration, as predicted by the model [8].

FIGS. 4A-C. Protein synthesis and diffusion in a single compartment. (A) Fluorescence image of a compartment. The hole at the edge of the capillary (left) seems bright due to cell extract auto-fluorescence and the integration of the signal along the line of sight of the entire length of the hole. (B) GFP profile along the capillary at different time points indicated by color matching the colored time points in (C). the x-axis indicates the normalized distance from the hole. (C) dynamics of GFP inside the compartment as function of time.

FIGS. 5A-E. Dependence of expression on the compartment geometry. (A) fluorescence image of gene expression on the chip at steady state. (B) typical compartment dynamics as function of time for different lifetimes. (C,D) calculated onset time and steady state amplitude (respectively) as function of lifetime, averaged over different parameters combinations. Linear relations are apparent. (E) steady state amplitude as function of lifetime for different through-hole radii. No significant effect observed.

FIGS. 6A-C. Oscillator lifetime scan. (A) time-lapse images of fluorescence on the chip (holes have been artificially removed from images for clarity). (B) typical compartment dynamics for different lifetimes. (C) average oscillation period for compartments as function of the calculated lifetime. Values obtained from solving the numerical model shown in red.

FIGS. 7A-D. localized perturbation at the center. (A) sketch of the compartment geometry. The compartments marked with orange (25) have longer intrinsic period (τ₀=20 min), while the rest of the compartments (875) have shorter intrinsic period (τ₀=10 min). (B) space-time plot of the compartments on the diagonal of the array. The boundary effect between the oscillator populations is evident (pink color). (C) averaged protein dynamics in compartments grouped by the distance from edge. (D) time lapse image sequence of GFP expression levels on the chip.

FIGS. 8A-C. Collective behavior in the pseudo 1D configuration. (A) sketch of the compartment geometry. The compartments marked with orange (90) have longer intrinsic period (τ₀=20 min), while the rest of the compartments (810) have shorter intrinsic period (τ₀=10 min). (B) peak time of the second oscillation for each compartment in the array (C) time lapse image sequence of GFP expression levels on the chip. Time is indicated by the arrows (from left to right and top to bottom).

FIGS. 9A-C. Spatial patterns driven by two topological perturbations. (A) sketch of the compartment geometry. The compartments marked with orange (50) have longer intrinsic period (τ₀=20 min), while the rest of the compartments (850) have shorter intrinsic period (τ₀=10 min). (B) oscillation frequency per compartment for 4 consecutive oscillations (from left to right: 2^{nd} to 5^{th}). The time dependent frequency is calculated by taking the inverse of the time difference between consecutive peaks. (C) time lapse image sequence of GFP expression levels on the chip. Time is indicated by the arrows.

FIG. 10 illustrates an experimental setup and data acquisition according to embodiments of the invention. The sealed silicon device placed in a custom made chip holder. The holder is placed inside thermally stable incubation chamber located on the translational stage (X-Y) of inverted microscope, and cell extract is flown with constant rate via external tubing. Fluorescent microscopy is used to image the GFP gene expression reporter (488 nm wavelength).

FIG. 11 is an image of automated acquisition of data for each compartment, from the raw data recorded during the experiment.

FIG. 12 is a diagram illustrating a microfluidic device according to embodiments of the invention.

FIG. 13 is a diagram illustrating a microfluidic device according to embodiments of the invention.

FIG. 14 is a diagram illustrating a microfluidic device according to embodiments of the invention.

FIG. 15 is a diagram illustrating a microfluidic device according to embodiments of the invention.

FIG. 16 is a diagram illustrating a microfluidic device according to embodiments of the invention.

### DESCRIPTION OF SPECIFIC EMBODIMENTS OF THE INVENTION

The present invention, in some embodiments thereof, relates to a microfluidic device for analyzing a plurality of biological/chemical reactions at steady-state.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Understanding how cell-cell local diffusive interactions driven by gene circuits lead to large-scale self-organization and complex collective behavior is an experimental challenge because many processes are entangled in a living organism. The present inventors postulated that development of minimal model systems of communicating artificial cells on a chip based on cell-free gene-expression could enable studying fundamental processes in multicellular systems such as morphogenesis. Programmable biochip devices hold promise as autonomous systems capable of biological computation.

The present inventors fabricated and assembled a chip of 2D communicating DNA compartments as artificial cells communicating on a chip. The system is based on a 3D silicon microfluidic device comprising a laminar flow layer that feeds a protein synthesis reaction into a quasi-2D diffusive layer of compartments.

Whilst reducing the present invention to practice, the present inventors characterized an exemplary device and demonstrated integration of a plurality of reaction chambers capable of long-term gene-expression. Coupled 2D arrays were designed with control over the genetic program of each compartment, the geometry, as well as the array topology and coupling strength. The present inventors validated experimentally a linear model of each reaction chamber capable of steady-state gene-expression reactions, and established a 2D reaction-diffusion scenario, which allows programming communication and spatiotemporal patterns.

Microfluidic devices described herein comprise at least one feeding channel and a plurality of reaction chambers. The feeding channel is situated on a different plane to the reaction chambers of the device (e.g. above or beneath). This allows for fabrication of a device having a network of interconnecting reaction chambers (e.g. each reaction chamber may be connected to 3, 4, 5, 6 or more reaction chambers. Devices containing more than 100 reaction chambers on one surface are thus contemplated. By having the feeding channel on a different plane to the reaction chambers, there is a high degree of freedom with respect to the spatial organization of the reaction chambers.

Microfluidic devices generated according to the teachings of the present invention can be employed in a myriad of microfluidic applications including various chemical and biochemical analyses and syntheses, both for preparative and analytical applications.

Thus, according to one aspect of the present invention there is provided a microfluidic device. The microfluidic device comprises:
(i) a first surface which comprises a plurality of reaction units, each reaction unit having a test chamber connected to at least one opening via a feeding capillary, wherein the at least one opening is in communication with a hollow along a depth of the device, the hollow being defined by internal walls; and
(ii) a second surface which comprises at least one flow-through channel having at least one inlet port and at least one outlet port,
wherein the hollow extends to the second surface and fluidly connects the test chamber with the flow-through channel.

As used herein the phrase “microfluidic device” refers to a synthetic device in which minute volumes of fluids are flowed. The flow channel is generally fabricated at the micron to sub-micron scale, e.g., the flow-through channel typically has at least one cross-sectional dimension in the range of less than about 1 mm. Microfluidic devices of the present invention can be incorporated in complicated systems such as those described herein below.

The device is made from a substrate (e.g. silicon or glass) having two opposing surfaces (e.g. an upper surface and a lower surface). The substrate may be made from a single material or a combination of materials.

One of the surfaces of the substrate is fabricated to include a flow-through channel. Typically, the flow-through channel is carved out of (e.g. etched from) one of the surfaces (e.g. the bottom surface, when in use) of the substrate.

The term “flow-through channel” as used herein, refers to a low resistance flow channel, about 20 microns to about 250 microns deep, preferably about 50 microns to about 200 microns deep and more preferably about 100 microns to about 200 microns deep, e.g. about 150 microns deep. Flow-through channels are sufficiently wide (perpendicular to the direction of flow) to not inhibit the flow of fluid through the channel, and not excessively wide to inhibit the function of optional valves. Such considerations are well understood by those of ordinary skill in the art. Exemplary widths of the flow-through channel are between 50 microns-1 mm wide, e.g. about 100 microns. The flow-through channel has at least one inlet port and at least one outlet port, at least one of which being in fluid communication with a reservoir such as by tubing. Fluids may be passively or actively infused into the flow channels such as by capillary forces or pumps (e.g., external pumps, e.g., peristaltic pumps or electro-osmotically pumps).

Liquid flow through the flow-through channel may be regulated using a valve.

A “valve” is a component of a device that regulates flow through the flow channel of the device by substantially inhibiting (or reducing) flow upon closure. Substantially inhibiting the flow means that flow is inhibited at least 80%, preferably at least 90%, more preferably at least 95%, even more preferably at least 99%, most preferably flow is completely (i.e., 100%) inhibited. The size of the valve is dependent on the size and shape of the flow-through channel and the amount of pressure required to close the valve. In a preferred method, the flow-through channel is about 250 microns wide and the valve is about 300 microns wide. The flow-through channel and control valve cross perpendicularly. Upon actuation of the valve, preferably by hydrostatic pressure, the channel closes and opens.

One of the surfaces of the substrate is constructed to include at least two reaction units. A single reaction unit typically comprises a test chamber, a feeding capillary and an opening. Typically, the reaction units are carved out of (e.g. etched from) a surface of the substrate which is opposite the surface which comprises the flow-through channel.

As used herein, the term “test chamber” refers to a compartment of the reaction unit in which a test reaction takes place. The test chamber is connected to an opening via a feeding capillary. The test chamber may be any shape—e.g. rectangular, square or circular. According to one embodiment, the test chamber is circular and has a diameter of about 20-200 microns or more typically between 20-100 microns. Typically, the depth of the test chamber is about 1-3 microns.

The depth ratio of the reaction unit: flow-through channel is greater than 1:5, 1:10 or even 1:20.

In further embodiments, the rate of flow in the test chamber is less than about 10% than that in the direction of fluid flow in the flow-through channel. In a preferred embodiment, the rate of flow in the test chamber is less than about 5%, more preferably less than about 1%, most preferably less than about 0.1% of the flow rate in the flow-through channels in the device.

The test chambers typically hold a volume of less than 100 pl, in other instances less than 50 pl; in other instances less than 40 pl, 30 pl, 20 pl or 10 pl.

The term “feeding capillary” as used herein, refers to a high resistance channel, which connects the test chamber of the reaction unit to the opening of the reaction unit. The feeding capillary is typically about 1 micron to about 50 microns deep, more preferably about 1 micron to about 20 microns deep, more preferably about 1 micron to about 10 microns deep. The length of the feeding capillary can vary between 20 microns to about 1 mm or between 20 microns to about 500 microns. The width of the feeding capillary is typically between 1-50 microns. According to embodiments of the present invention the ratio of the width of feeding capillary: width of the flow-through channel is greater than 1:3, and more preferably greater than 1:5. Exemplary ratios include 1:5, 1:6, 1:7, 1:8, 1:9, 1:10 and 1:20. The depth of the test chamber may be identical to the depth of the feeding capillary. Alternatively, the test chamber and feeding capillary may be of different depths. In one embodiment, the depth of the feeding capillary is no more than 10 times the depth of the test chamber.

It will be appreciated that when the surface of the device comprises more than one reaction unit, the length, width or depth of the feeding capillary of the first reaction unit may be identical to the length of the feeding capillary of the second reaction unit or non-identical.

According to a particular embodiment, the hydrodynamic resistance of the feeding capillary is at least 3, 4, 5 or 6 orders of magnitude higher than in the flow-through channel. This reduces the flow in the feeding capillary by 3, 4, 5 or 6 orders of magnitude compared with the flow in the flow chamber.

Formulae for determining hydrodynamic resistance of capillaries and holes are provided in the Examples section, herein below.

Each test chamber is connected to the opening by at least one feeding capillary so as to allow diffusion of molecules from the flow-through channel to the test chamber of the reaction unit and vice versa. The feeding capillary according to embodiments of this aspect of the present invention is essentially perpendicular to the flow-through channel.

The opening of the reaction unit can be of any shape, e.g. round (e.g. having a diameter between 5-50 microns), square, rectangular etc. The opening is in communication with a hollow along a width of the device, wherein the hollow is defined by internal walls. The hollow therefore creates a through-hole, and provides a means for molecules which flow through the flow-through channel to diffuse into the reaction unit and vice versa.

The relative dimensions of the at least one flow-through channel, the feeding capillary and the at least one opening being of dimensions allow diffusion of a molecule from the flow-through channel to the test chamber of the reaction unit via the hollow and vice-versa and prevent (or reduce to a negligible amount) flow of fluid in the reaction unit. Thus, the rate of movement of a molecule through the reaction chamber via diffusion is at least 10, 20, 50, 100 or more the rate of movement of the molecule (e.g. via convection) through the reaction chamber via fluid flow.

The relative dimensions of the hollow and test chamber are such that the volume of liquid held in the hollow is typically between about 3-10 times the volume of liquid held in the test chamber.

As mentioned, the device of the present invention comprises at least two reaction units. According to one embodiment, the two reaction units are connected via at least one interconnecting capillary which allows diffusion of molecules between one reaction unit to another. Preferably, a test chamber of a first of the two reaction units is connected to a test chamber of a second of the two reaction units via the interconnecting capillary. The device may comprise a plurality of interconnecting capillaries, depending on the number of reaction units on the device and the particular design. The length, width or depth of the interconnecting capillaries on the device may be identical or non-identical.

According to a particular embodiment, the hydrodynamic resistance of the interconnecting capillary is at least 3, 4, 5 or 6 orders of magnitude higher than in the flow-through channel. This reduces the flow in the feeding capillary by 3, 4, 5 or 6 orders of magnitude compared with the flow in the flow chamber.

In one embodiment, the width of the interconnecting capillary is essentially identical to the width of the feeding capillary. In another embodiment, the width of the interconnecting capillary is narrower than the width of the feeding capillary. Thus, for example the width ratio between the interconnecting capillary and the feeding capillary may be 1:1, 1:2, 1:3 or more.

The microchannel length between the two reaction units determines the delay time between the synthesis of a protein in one compartment and the time it reaches the 2^{nd} compartment. Thus, the capillary length controls a delay time that can be used for example to create a temporal gene expression pulse.

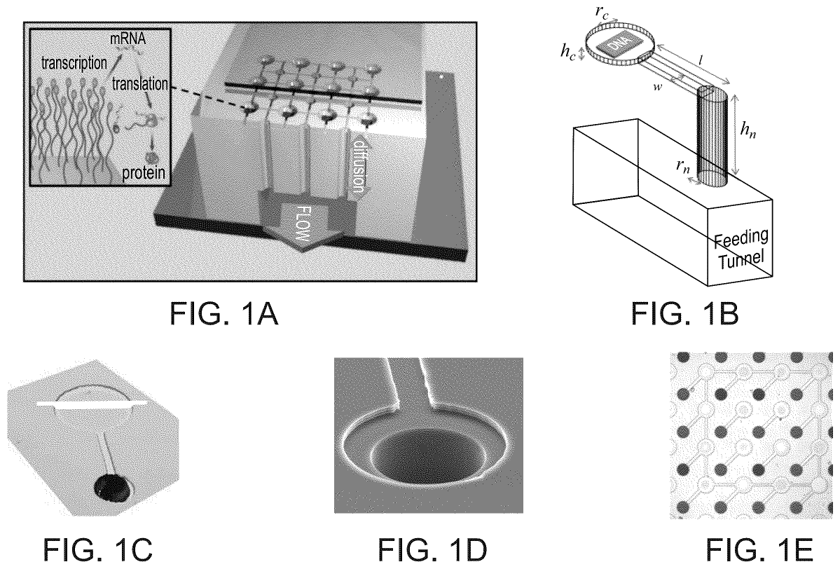

The device of the present invention can comprise any number of reaction units e.g. 2-1000, 10-500. When there is a large number of reaction units (e.g. over ten), the reaction units may be arranged on the surface of the substrate as a grid having columns and rows. In one embodiment, the device comprises a plurality of reaction units arranged in a grid such that at least one test chamber of a reaction unit is connected to, two, three four or more test chambers of corresponding reaction units via interconnecting capillaries. This is illustrated in FIG. 1E. In one embodiment, the reaction units are aligned such that each of the openings are organized in a straight line which is in a parallel orientation to a single flow-channel on the opposing face of the device.

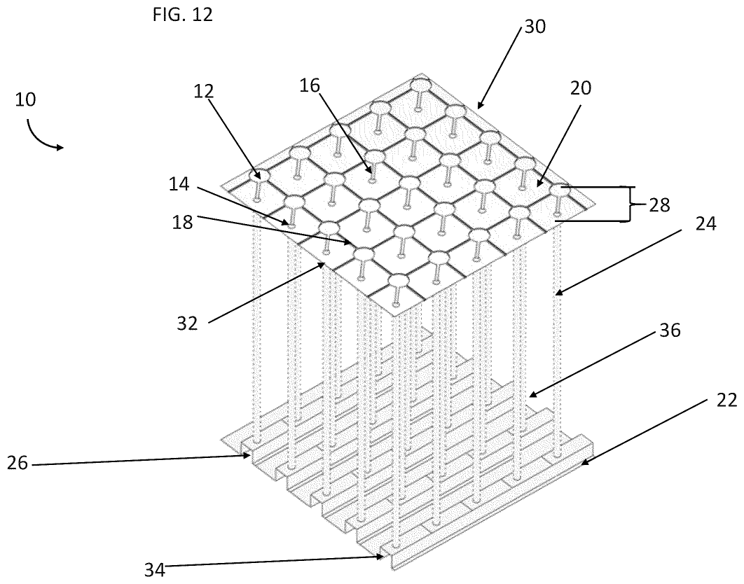

FIG. 12 presents an exemplary device 10 having a surface 20 (e.g. top surface) which comprises a plurality of test chambers 12, each test chamber 12 being connected to an opening 14 via a feeding capillary 16. Opening 14 are through-holes and comprise a hollow 24 which traverses the thickness w of the device. Reaction unit 28 comprises a test chamber 12, opening 14 and feeding capillary 16. The openings 14 may be aligned in a row which traverses a back side 30 of the device to the front side 32 of the device. In FIG. 12 two rows of reaction units 28 are illustrated, although it will be appreciated that the device 10 may comprise any number of rows of test chambers. Device 10 has a second surface 22 (e.g. bottom surface) which comprises a flow-through channel 26. Fluid enters the flow channel 26 from side 34 and exits the flow channel from side 36. The position of the flow channel 26 on surface 22 relative to the position of a row of reaction units 28 on surface 20 is such that openings 14 of reaction units 28 are directly above the flow channels 34. Each row of test chambers feeds into a single feeding channel, via openings 14.

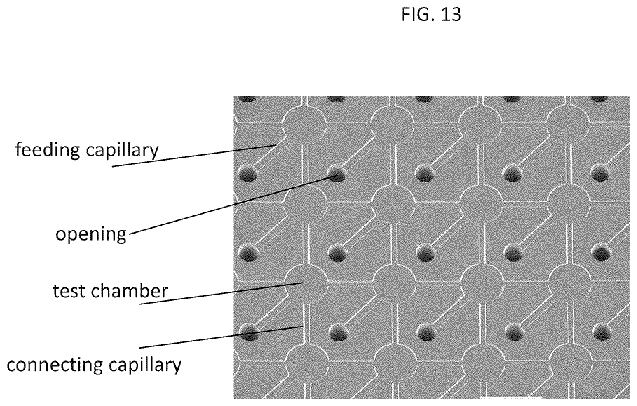

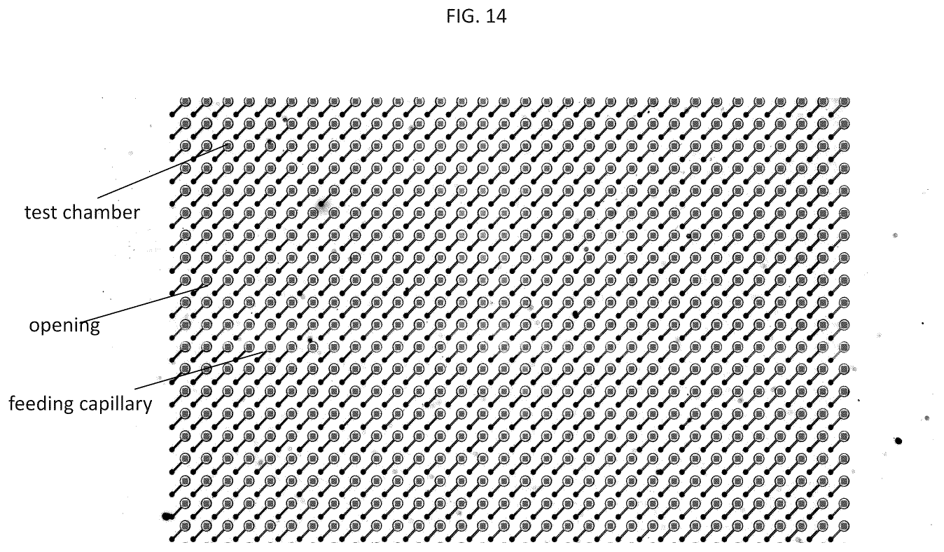

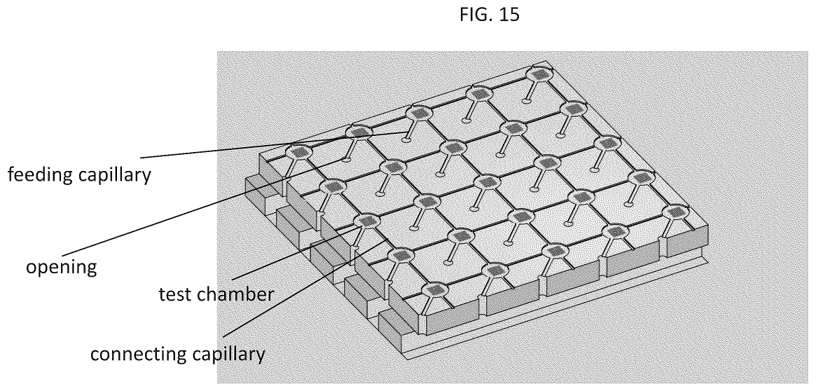

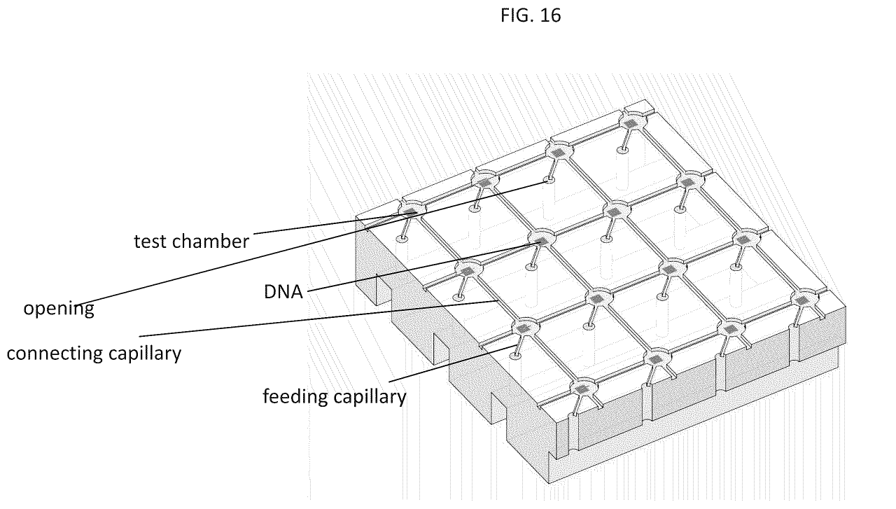

Other designs of the upper surface of exemplary devices are shown in FIGS. 13-16.

The device can be used to analyze biological, chemical reactions under steady state conditions, as further described herein below. To ensure steady state, the resistance of fluid flowing through the reaction unit is higher than the resistance in the flow-through channel. This resistance is typically established by having feeding and interconnecting capillaries that are substantially and sufficiently shallower and/or narrower than the flow-through channels and openings and hollows of appropriate dimensions. This ensures that there is essentially no liquid flow in the reaction unit and/or through the hollow (except during the initial set-up as further described below). Movement of molecules present in the fluid from the flow-through channel to the reaction unit and vice versa occurs essentially via diffusion only. Size parameters of the components of the reaction units, hollows and flow-through channels can be readily determined by one of ordinary skill in the art using mathematical or empirical modeling. Exemplary size parameters are provided herein above and under.

In one embodiment, the depth ratio of a reaction unit:flow-through channel is greater than 1:5. Other exemplary ratios include 1:5, 1:6, 1:7, 1:8, 1:9, 1:10 and 1:20.

By altering the dimensions of the feeding capillary and the flow-channel, the τ_{flow/}τ_{diffusion} can be controlled. Thus, according to one embodiment, the τ_{flow/}τ_{diffusion} is about 1%. According to another embodiment, the τ_{flow/}τ_{diffusion} can be about 0.1%.

In further embodiments, the rate of flow in the feeding capillary is less than about 10% than that in the direction of fluid flow in the flow-through channel. In a preferred embodiment, the rate of flow in the feeding capillary is less than about 5%, more preferably less than about 1%, most preferably less than about 0.1% of the flow rate in the flow-through channels in the device.

The device further comprises a cover layer (e.g., glass or plastics) sealed thereto, such that the cover layer forms one wall of the microfluidic path. Alternatively, the device once removed from the mother mold is sealed to a thin elastomeric membrane such that the flow path is totally enclosed in elastomeric material. The resulting elastomeric device can then optionally be joined to a substrate support.

According to one embodiment, the device is sealed using a gas permeable sealant such as PDMS. The sealing may be covalent or non-covalent. The device may also be sealed using a coated or non-coated coverslip. Exemplary coatings are further described herein below.

Devices of the present invention may be constructed utilizing single and multilayer soft lithography (MSL) techniques and/or sacrificial-layer encapsulation methods. The basic MSL approach involves casting a series of elastomeric layers on a micro-machined mold, removing the layers from the mold and then fusing the layers together. In the sacrificial-layer encapsulation approach, patterns of photoresist are deposited wherever a channel is desired. These techniques and their use in producing microfluidic devices is discussed in detail, for example, by Unger et al. (2000) Science 288:113-116, by Chou, et al. (2000) “Integrated Elastomer Fluidic Lab-on-a-chip-Surface Patterning and DNA Diagnostics, in Proceedings of the Solid State Actuator and Sensor Workshop, Hilton Head, S.C.; and in PCT Publication WO 01/01025.

Preferably, the substrate material is substantially non-fluorescent or emits light of a wavelength range that does not interfere with the photoactivation. Examples of such materials include, but are not limited to, silica-based materials (exemplified hereinbelow) and elastomeric materials.

The term “elastomer” and “elastomeric” as used herein refers to the general meaning as used in the art. Thus, for example, Allcock et al. (Contemporary Polymer Chemistry, 2nd Ed.) describes elastomers in general as polymers existing at a temperature between their glass transition temperature and liquefaction temperature. Elastomeric materials exhibit elastic properties because the polymer chains readily undergo torsional motion to permit uncoiling of the backbone chains in response to a force, with the backbone chains recoiling to assume the prior shape in the absence of the force. In general, elastomers deform when force is applied, but then return to their original shape when the force is removed. The elasticity exhibited by elastomeric materials can be characterized by a Young's modulus. The elastomeric materials utilized in the microfluidic devices disclosed herein typically have a Young's modulus of between about 1 Pa-1 TPa, in other instances between about 10 Pa-100 GPa, in still other instances between about 20 Pa-1 GPa, in yet other instances between about 50 Pa-10 MPa, and in certain instances between about 100 Pa-1 MPa. Elastomeric materials having a Young's modulus outside of these ranges can also be utilized depending upon the needs of a particular application. Examples of elastomeric materials which can be used to fabricate the devices of the present invention include, but are not limited to, GE RTV 615 (formulation), a vinyl-silane crosslinked (type) silicone elastomer (family e.g., PDMS).

The choice of materials typically depends upon the particular material properties (e.g., solvent resistance, stiffness, gas permeability, and/or temperature stability) required for the application being conducted. Additional details regarding the type of materials that can be used in the manufacture of the components of the microfluidic devices disclosed herein are set forth in Unger et al. (2000) Science 288:113-116, and PCT Publications WO 02/43615, and WO 01/01025. Exemplary low-background substrates include those disclosed by Cassin et al., U.S. Pat. No. 5,910,287 and Pham et al., U.S. Pat. No. 6,063,338.

Preferred elastomers of the instant invention are biocompatible, gas permeable, optically clear elastomers useful in soft lithography including silicone rubbers, most preferably PDMS. Other possible elastomers for use in the devices of the invention include, but are not limited to, polyisoprene, polybutadiene, polychloroprene, polyisobutylene, poly(styrene-butadiene-styrene), the polyurethanes, and silicone polymers; or poly(bis(fluoroalkoxy)phosphazene) (PNF, Eypel-F), poly(carborane-siloxanes) (Dexsil), poly(acrylonitrile-butadiene) (nitrile rubber), poly(1-butene), poly(chlorotrifluoroethylene-vinylidene fluoride) copolymers (Kel-F), poly(ethyl vinyl ether), poly(vinylidene fluoride), poly(vinylidene fluoride-hexafluoropropylene) copolymer (Viton), elastomeric compositions of polyvinylchloride (PVC), polysulfone, polycarbonate, polymethylmethacrylate (PMMA), and polytertrafluoroethylene (Teflon).

In a preferred embodiment, the substrate material is substantially non-reactive with nucleic acids, thus preventing non-specific binding between the substrate and the nucleic acids. Methods of coating substrates with materials to prevent non-specific binding are generally known in the art. Exemplary coating agents include, but are not limited to cellulose, bovine serum albumin, and poly(ethyleneglycol). The proper coating agent for a particular application will be apparent to one of skill in the art.

As mentioned, the device can be used to analyze biological, chemical reactions under steady state conditions.

For this, a component of the reaction (e.g. biomolecule) is typically attached to a surface of at least a portion of the test chamber.

The length of the feeding capillary and the amount of component attached to the surface determines the dilution time of end-products of the chemical/biological reaction in the reaction unit and therefore determines the steady state concentration of the end-products of the chemical/biological reaction.

The dimensions of the device are such that the end-products of the reaction in the reaction unit forms a gradient along the feeding capillary, wherein the concentration at where the component (e.g. DNA) is attached (for example at the test chamber) is the highest, gradually decreasing along the feeding channel such that the concentration of the end-product at the junction of the feeding capillary and opening is at its lowest. According to one embodiment, the end-product (e.g. RNA and/or protein) expressed in the reaction unit may create a linear or exponential profile.

According to one embodiment, the biomolecule is a component of a cellular process including but not limited to protein expression, protein assembly, protein modification. Examples of components of biological reactions that can be attached to the surface of the test chamber include proteins and/or nucleic acids. In one embodiment, the component is a cellular structure or a cell.

According to a particular embodiment, the surface of at least a portion of the reaction unit (e.g. test chamber) is attached to an isolated nucleic acid (e.g. DNA) and the process which is analyzed in the device is protein expression.

As used herein, the phrase “isolated nucleic acid” refers to a nucleic acid which is not comprised in or on a cell.

According to another embodiment, when the isolated nucleic acid is attached to the test chamber, it is devoid of cellular components (such as proteins, lipids etc.)

The nucleic acid may be single stranded or double stranded. The nucleic acid may be DNA (e.g. cDNA, genomic DNA, synthetic DNA), RNA, a combination of both. The nucleic acid may be isolated from a cell, or may by synthesized in vitro. Typically, the nucleic acids of this aspect of the present invention comprise at least one promoter and encode a polypeptide.

The nucleic acids may be of any length. According to a particular embodiment, the nucleic acids are between 200 bp-500 kbp, or between 200 bp-2000 kbp, or between 200 bp-100 kbp, or between 200 bp-40 kbp, or between 200-5000 bp.

Nucleic acids of this aspect of the present invention are further described herein below.

The nucleic acids of this aspect of the present invention are typically linear.

According to one embodiment, the surface of the test chamber (or portion thereof) is coated with nucleic acids.

According to another embodiment, the feeding capillary (or portion thereof) is coated with nucleic acids.

According to still another embodiment, the test chamber and the feeding capillary are coated with nucleic acids.

Preferably, the density of the nucleic acid on the substrate is between 10² DNA μm²-10⁵ DNA μm², for example in the order of 10² DNA μm².

The nucleic acid of the present invention is typically orientated on the substrate such that the regulatory region of the nucleic acid (e.g. promoter) is closer to the substrate and the polypeptide coding region is further from the substrate.

The isolated nucleic acids may be attached to the reaction unit (or portion thereof) in a wide variety of ways, as will be appreciated by those in the art. The nucleic acids may either be synthesized first, with subsequent attachment to the substrate, or may be directly synthesized on the substrate. The substrate and the nucleic acid may be derivatized with chemical functional groups for subsequent attachment of the two. For example, the substrate may be derivatized with a chemical functional group including, but not limited to, amino groups, carboxyl groups, oxo groups or thiol groups. Using these functional groups, the nucleic acid may be attached using functional groups on the nucleic acid either directly or indirectly using linkers.

The isolated nucleic acid may also be attached to the substrate non-covalently. For example, a biotinylated nucleic acid can be prepared, which may bind to surfaces covalently coated with streptavidin, resulting in attachment. Alternatively, a nucleic acid may be synthesized on the surface using techniques such as photopolymerization and photolithography. Additional methods of attaching nucleic acids to solid surfaces and methods of synthesizing nucleic acids on substrates are well known in the art, i.e. VLSIPS technology from Affymetrix (e.g., see U.S. Pat. No. 6,566,495, and Rockett and Dix, “DNA arrays: technology, options and toxicological applications,” Xenobiotica 30(2):155-177, all of which are hereby incorporated by reference in their entirety).

According to a preferred embodiment of this aspect of the present invention, the substrate is coated with a coat composed of a compound which can be represented by the general formula I below:

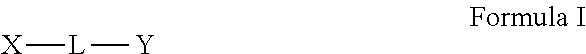

wherein X is the functionalized group capable of binding to a substrate; L is the polymer capable of forming a monolayer on a substrate; and Y is a photoactivatable group capable of generating a reactive group upon exposure to light.

The functionalized group is preferably selected such that it binds to the substrate by reacting with at least one functional group present on a surface of a substrate.

Preferred functionalized groups according to the present invention comprise one or more reactive silyl group(s).

As used herein, the phrase “reactive silyl group” describes a residue of a compound comprising at least one silicon atom and at least one reactive group, such as an alkoxy or halide, such that the silyl group is capable of reacting with a functional group, for example on a surface of a substrate, to form a covalent bond with the surface. For example, the reactive silyl group can react with the surface of a silica substrate comprising surface Si—OH groups to create siloxane bonds between the compound and the silica substrate.

Exemplary reactive silyl groups that are usable in the context of the present invention include, without limitation, trialkoxysilanes, alkyldialkoxysilanes, alkoxydialkylsilanes, trihalosilanes, alkyldihalosilanes and dialkylhalosilanes. Such reactive groups are easily reacted when contacted with free hydroxyl groups on a surface of solid surfaces and particularly with such hydroxyl groups on a silica surface.

Herein, the terms “silica” and “SiO₂” are used interchangeably.

In a preferred embodiment of the present invention the reactive silyl group is trialkoxysilane such as, for example trimethoxysilane, triethoxysilane, tripropyloxysilane or trihalosilane such as, for example, trichlorosilane.

The functionalized group according to the present invention may further include a chemical moiety that is terminated with the reactive silyl group. Such a chemical moiety can comprises, for example, alkyl, alkenyl, aryl, cycloalkyl and derivatives thereof, as these terms are defined herein.

Preferably, the functionalized group comprises an alkyl terminating with a trialkoxysilane.

As discussed hereinabove, the polymer is selected so as to form a monolayer on the substrate. Thus, the polymer group in the compounds of the present invention may be any hydrophobic, hydrophilic and amphyphilic polymer that has suitable characteristics for forming a monolayer. Such characteristics include, for example, long, relatively inert chains, which may interact therebetween via e.g., hydrogen or Van-der-Waals interactions.

A preferred polymer according to the present invention comprises polyethylene glycol (PEG). As described hereinabove, PEG is characterized by resistance to nonspecific absorptions of biomolecules and is therefore beneficial for use in some contexts of the present invention. In addition, when self-assembled on a substrate, PEG chains typically interact therebetween via hydrogen bonds, so as to produce a well-ordered monolayered film.

The polyethylene glycol residue in the compounds of the present invention can be derived from PEGs having a molecular weight that ranges from about 400 grams/mol and about 10000 grams/mol. Preferred PEGs are those having a molecular weight that ranges from about 2000 grams/mol and about 5000 grams/mol. Such PEGs allow the productions of a monolayered film when deposited on a solid surface in the presence of a functionalized group, as described hereinabove.

The polyethylene glycol residue may be substituted or unsubstituted and can be represented by the general Formula II below:

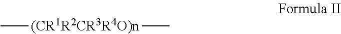

wherein n is an integer from 10 to 200; and R¹, R², R³ and R⁴ are each independently selected from the group consisting of hydrogen, alkyl, cycloalkyl, aryl, alkenyl alkynyl, alkoxy, thioalkoxy, aryloxy and thioaryloxy.

In a preferred embodiment, the PEG is unsubstituted such that R¹, R², R³ and R⁴ are each hydrogen.

In another preferred embodiment, the PEG residue is a medium-sized residue such that n is an integer from 60 to 100.

The polymer is preferably attached to the functionalized group described above via a linking moiety.

Exemplary linking moieties include, without limitation, oxygen, sulfur, amine, amide, carboxylate, carbamate, sulphonate, sulphonamide, phosphate, hydrazine, hydrazide, as these terms are defined herein and derivatives thereof.

In a representative example the linking moiety is an amide, formed between a carboxylic end group of the polymer and an amine end group of the functionalized moiety, as is detailed herein under.

The compounds of the present invention, by comprising the functionalized group and the polymer described hereinabove, readily form self-assembled monolayers when contacted with a substrate, in a one-step, simple to perform, reaction.

As the polymer residue in the compounds of the present invention further has a photoactivatable group attached thereto, each of the formed monolayers has a photoactivatable group attached thereto.

As used herein, the phrase “photoactivatable group” describes a group that is rendered active when exposed to photoactivation, namely when exposed to light. Photoactivatable groups typically comprise a protected reactive group, which upon exposure to light are de-protected, so as to generate a reactive group.

As used herein, the phrase “reactive group” describes a chemical moiety that is capable of interacting with another moiety. This interaction typically results in a bond formation between these moieties, whereby the bond can be, for example a covalent bond, a hydrogen bond, a coordinative bond, or an ionic bond.

Representative examples of reactive groups include, without limitation, amine, hydroxy, thiohydroxy, halo, alkoxy, thioalkoxy, aryloxy, thioaryloxy, carboxylate, phosphate, phosphonate, sulfate and sulfonate, as these terms are defined herein.

Depending on the intended use of the compound, the photoactivatable group is selected so as to generate a desired reactive group Thus, for example, a photoactivatable group that comprises a carbamate can generate upon exposure to light amine as the reactive group.

The photoactivatable groups according to the present invention are preferably derived from photoactivatable compounds and therefore preferably include a residue of, for example, photoactivatable compounds that has light-absorbing characteristics such as 6-nitrovertaryl chloroformate, 6-nitrovertaryl carbonyl, 2-nitrotoluene, 2-nitroaniline, phenacyl, phenoxy, azidoaryl, sulfonic ester, desyl, p-hydroxyphenacyl, 7-methoxy coumarin, o-ethylacetophenone, 3,5-dimethylphenacyl, dimethyl dimethoxybenzyloxy carbonyl, 5-bromo-7-nitroindolinyl, o-hydroxy-α-methyl cinnamoyl and 2-oxymethylene anthraquinone.

When exposed to light such as, for example, UV, IR, or visible light or a monochromatic light of a predetermined wavelength, reactive groups, which are capable of nucleic acids, as is detailed hereinunder, are generated.

The above-described compounds can be readily prepared using a simple two-steps synthesis. A process of preparing the compounds is described in details in PCT Application No. WO2006/064505 to the present inventor.

As discussed hereinabove, the substrate and the compound of the present invention are selected such that upon contacting the polymer with the substrate, a self-assembled monolayered film of the polymer forms on the substrate surface, in a one-step reaction.

The contacting procedure is preferably effected by incubating the compound of the present invention with the selected substrate, preferably in the presence of an organic solvent such as, for example, toluene.

Once a monolayered film of the polymer is deposited on the substrate surface, the reactive group for binding a screenable moiety can be generated by exposing a pre-selected area of the substrate to light.

Depending on the selected photoactivatable group and the active wavelength in which it is active, the light can be a UV, IR or visible light, or, optionally and preferably, the light can be a monochromatic light of a predetermined wavelength.

Exposure of a limited area of the substrate to light is preferably effected using a photo mask to illuminate selected regions the substrate and avoid coating the substrate at the periphery. However, other techniques may also be used. For example, the substrate may be translated under a modulated laser or diode light source. Such techniques are discussed in, for example, U.S. Pat. No. 4,719,615 (Feyrer et al.), which is incorporated herein by reference. In alternative embodiments a laser galvanometric scanner is utilized. In other embodiments, the synthesis may take place on or in contact with a conventional liquid crystal (referred to herein as a “light valve”) or fiber optic light sources. By appropriately modulating liquid crystals, light may be selectively controlled so as to permit light to contact selected regions of the substrate. Alternatively, synthesis may take place on the end of a series of optical fibers to which light is selectively applied. Other means of controlling the location of light exposure will be apparent to those of skill in the art.

The substrate may be irradiated either in contact or not in contact with a solution and is, preferably, irradiated in contact with a solution. The solution may contain reagents to prevent the by-products formed by irradiation. Such by-products might include, for example, carbon dioxide, nitrosocarbonyl compounds, styrene derivatives, indole derivatives, and products of their photochemical reactions. Alternatively, the solution may contain reagents used to match the index of refraction of the substrate. Reagents added to the solution may further include, for example, acidic or basic buffers, thiols, substituted hydrazines and hydroxylamines, or reducing agents (e.g., NADH).

In an exemplary embodiment, exposing the substrate to light is effected so as to provide a patterned substrate in which reactive groups are generated according to a pre-selected pattern. The pattern can be printed directly onto the substrate or, alternatively, a “lift off” technique can be utilized. In the lift off technique, a patterned resist is laid onto the substrate or onto the light source. Resists are known to those of skill in the art. See, for example, Kleinfield et al., J. Neurosci. 8:4098-120 (1998). In some embodiments, following removal of the resist, a second pattern is printed onto the substrate on those areas initially covered by the resist; a process that can be repeated any selected number of times with different components to produce an array having a desired format.

Once the reactive group is generated the device is preferably sealed using methods which are well known in the art. Low fluorescence adhesives which provide sealing and cover constructions are preferably used. Such adhesives are dimensionally stable and do not flow into microfluidic channels. They adhere to the cover layer without creating voids or gaps that may allow migration of components from one path to adjacent path, and they exhibit good stability to moisture and temperature change. Adhesives used in accordance with the present invention can be either flexible or rigid, but should preferably be clear and colorless (such adhesives can be obtained from Adhesives Research Inc.). Other adhesives include, but are not limited to, pressure sensitive adhesives, such as ethylene-containing polymers, urethane polymers, butyl rubber, butadiene-acrylonitrile polymers, butadiene-acrylonitrile-isoprene polymers, and the like. See, for example, U.S. Pat. No. 5,908,695 and references cited therein.

Binding the nucleic acid can be effected by directly attaching the moiety to the reactive group.

Alternatively, binding the nucleic acid is effected via a mediating moiety. As used herein, the phrase “mediating moiety” describes a mediating agent or a plurality of mediating agents being linked therebetween that may bind to both the reactive group and the screenable moiety and thus mediate the binding of the nucleic acid to the reactive group.

The mediating moiety can thus be a bifunctional moiety, having two reactive groups, each independently capable of reacting with the reactive group attached to the substrate or the screenable moiety. Alternatively, the mediating moiety can comprise two or more moieties, whereby the first moiety can be attached to the reactive group and to a second mediating moiety, whereby the second mediating moiety can bind the nucleic acid.

Optionally and preferably, the mediating moiety comprises an affinity pair, such as, for example, the biotin-avidin affinity pair. The biotin-avidin affinity pair is highly useful for integrating nucleic acids on the substrate. Additional affinity pairs are further described herein below.

Alternatively, the mediating moiety can comprise biotin. When attached to the reactive group, biotin can bind a variety of chemical and biological substances that are capable of reacting with the free carboxylic group thereof.

According to aspects of the present invention, the sequence of at least one of the isolated nucleic acids which is attached to the reaction unit (or portion thereof) encodes a promoter which is operatively linked to a nucleic acid sequence encoding a polypeptide.

As used herein, the term “promoter” refers to a nucleic acid fragment that functions to control the transcription of one or more genes, located upstream with respect to the direction of transcription of the transcription initiation site of the gene, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter.

A “constitutive” promoter is a promoter that is active under most environmental and developmental conditions. An example of a constitutive promoter is cytomegalovirus (CMV) or Rous sarcoma virus (RSV) promoter.

An “inducible” promoter is a promoter that is active under environmental or developmental regulation.

Examples of inducible promoters include the tetracycline-inducible promoter (Srour, M. A., et al., 2003. Thromb. Haemost. 90: 398-405), an IPTG inducible promoter, P70, P70_{b}, P₂₈, P₃₈ or Plac\arac (Pₗₐ).

In the isolated nucleic acid, the promoter is preferably positioned approximately the same distance from the heterologous transcription start site as it is from the transcription start site in its natural setting. As is known in the art, however, some variation in this distance can be accommodated without loss of promoter function.

A DNA segment such as an expression control sequence is “operably linked” when it is placed into a functional relationship with another DNA segment. For example, a promoter or enhancer is operably linked to a coding sequence if it stimulates the transcription of the sequence. DNA for a signal sequence is operably linked to DNA encoding a polypeptide if it is expressed as a pre-protein that participates in the secretion of the polypeptide. Generally, DNA sequences that are operably linked are contiguous, and, in the case of a signal sequence, both contiguous and in reading phase. However, enhancers need not be contiguous with the coding sequences whose transcription they control. Linking is accomplished by ligation at convenient restriction sites or at adapters, linkers, or PCR fragments by means know in the art.

According to one embodiment, the promoter is a eukaryotic promoter.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

According to another embodiment, the promoter is a prokaryotic promoter.

According to yet another embodiment, the promoter is a plant-specific promoter.

According to still another embodiment, the promoter is a tissue specific promoter.

The nucleic acid of this aspect of the present invention may further comprise an enhancer element. Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for some embodiments of the invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

Polyadenylation sequences may also be present in the nucleic acids in order to increase the efficiency of mRNA translation. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for some embodiments of the invention include those derived from SV40.

The nucleic acid of some embodiments of the invention can further include additional polynucleotide sequences that allow, for example, the translation of several proteins from a single mRNA such as an internal ribosome entry site (IRES) and sequences for genomic integration of the promoter-chimeric polypeptide.

In a particularly preferred embodiment of the invention, the nucleic acid molecule comprises a coding sequence coding for a predetermined amino acid sequence that is to be expressed.

According to one embodiment, at least one of the nucleic acids attached to the reaction unit encodes a transcription factor, an activator or a repressor.

According to another embodiment, at least one of the nucleic acids attached to the reaction unit encodes a polypeptide comprising a detectable moiety.

According to still another embodiment, the polypeptides are fluorescent polypeptides. Examples of such include, but are not limited to green fluorescent protein from Aequorea victoria (“GFP”), the yellow fluorescent protein and the red fluorescent protein and their variants (e.g., Evrogen).

According to still another embodiment, the polypeptides are phosphorescent polypeptides, chemiluminescent polypeptides or luminescent polypeptides.

It will be appreciated that a single reaction unit may be attached to isolated nucleic acids each having the same sequence. Alternatively, a single reaction unit may be attached to a plurality of isolated nucleic acids having different sequences. For example, a single reaction unit may be attached to a plurality of isolated nucleic acids encoding a transcriptome.

When the device comprises more than one test chamber, the present invention further contemplates attaching nucleic acids having a first sequence to the first test chamber and nucleic acids having a second sequence to the second test chamber.

Additional immobilizing agents may be attached to the surface of the test chamber so as to capture products of the chemical/biological reaction, as further described herein below. In one embodiment, the immobilizing agent captures a single product of the chemical/biological reaction. Once the product is captured, the product itself may serve as an immobilizing agent such that other products of different chemical/biological reactions are then captured. The product thus serves as an assembly agent for additional components. For example, when the biological process is protein expression, a single protein may be captured using an immobilizing agent which specifically binds to the protein (e.g. antibody). The protein may then bind to additional proteins which are products of other protein expression reactions. Alternatively, the additional proteins may be flowed through the flow-through channel and diffuse to the surface on which the first protein is captured. In this way a proteinaceous complex may be assembled at the protein product capture site.

The term “assembling” refers to the ability to generate a proteinaceous complex from its constituent subunits.

As used herein, the phrase “proteinaceous complex” refers to a collection of proteins which are bound together (either directly, or non-directly). In one embodiment, the proteins of the complex are bound to one another in their natural state. In one embodiment, at least some of the proteins of the complex are associated via at least one RNA. In another embodiment, the proteins are associated non-covalently (either directly with one another, or non-directly through additional components of the complex, such as via RNA) to generate a functional complex—for example a functional ribosomal subunit or a functional ribosome.

In the case of a ribosomal subunit, the ribosomal proteins are associated with the ribosomal RNA non-covalently by multiple weak interactions, including electrostatic and Van-der-Waals interaction. The ribosomal RNA is a scaffold for the assembly of the ribosomal subunits. Ribosomal proteins bind to the ribosomal RNA in a hierarchal order, some bind independently of others, some bind contingently on the pre-binding of others.

As used herein, the term “functional ribosome” refers to a ribosome that is capable of linking amino acids together in the order specified by messenger RNA (mRNA) molecules.

The term “functional ribosomal subunit” refers to a subunit, which together with other subunits of the ribosome, is capable of forming a functional ribosome.

Additional examples of proteinaceous complexes include, but are not limited to a bacteriophage, a spliceosome, a proteasome, a replisome, a divisome, a virus and a proteasome subunit.

In one embodiment, the proteinaceous complex is a eukaryotic (e.g. mammalian or yeast) proteinaceous complex.

In still another embodiment, the proteinaceous complex is a human proteinaceous complex.

In another embodiment, the proteinaceous complex is a prokaryotic (e.g. bacterial) proteinaceous complex.

According to a particular embodiment, the proteinaceous complex is a bacterial ribosomal subunit—e.g. an E. Coli ribosomal subunit or an S. Aureus ribosomal subunit.

According to a more particular embodiment, the proteinaceous complex is an E. Coli small ribosomal subunit or an S. Aureus small ribosomal subunit.

The proteinaceous complex of this aspect of the present invention comprises at least 2 proteins, at least 3 proteins, at least 4 proteins, at least 5 proteins, at least 6 proteins, at least 7 proteins, at least 8 proteins, at least 9 proteins, at least 10 proteins, at least 11 proteins, at least 12 proteins, at least 13 proteins, at least 14 proteins, at least 15 proteins, at least 16 proteins, at least 17 proteins, at least 18 proteins, at least 19 proteins, at least 20 proteins.

The proteinaceous complex of this aspect of the present invention may comprise between 5-20 proteins, 5-10 proteins, 10-30 proteins, 20-40 proteins or 30-50 proteins.

The proteinaceous complex of this aspect of the present invention may comprise additional components other than proteins including for example polynucleotides (such as DNA or RNA molecules). In a particular embodiment, the proteinaceous complex comprises protein and RNA. In still another embodiment, the proteinaceous complex consists of protein and RNA.

In one embodiment, the immobilizing agent is an antibody directed towards the complex.

In one embodiment, the immobilizing agent is an antibody directed towards an affinity tag pair which is expressed with the component of the complex.

The immobilizing agent (e.g. antibody) may comprise an affinity tag itself (e.g biotin) so that it can be immobilized to the chamber.

Affinity tags and affinity tag pairs are further described herein below.

Immobilization of the immobilizing agent can be effected as known in the art. In one embodiment, the immobilizing agent (e.g. antibody) is biotinylated, mixed with streptavidin and attached to the surface of the chamber as described above for biotinylated nucleic acids (see also examples section herein below). Preferably, the immobilizing agent is immobilized onto the surface of the chamber in a predefined continuous pattern surrounding the nucleic acids, which are themselves immobilized. The pattern may comprise of any shape—e.g. circle, square, hexagon, pentagon etc.

Preferably, the immobilizing agent is immobilized onto the identical surface of the reaction chamber onto which the nucleic acids are immobilized.

Preferably, the pattern of the immobilized immobilizing agent is surrounding the nucleic acids which are immobilized to the same surface.

According to a particular embodiment, at least 50% of the surface of the reaction chamber is patterned with the immobilizing agent.

According to a particular embodiment, at least 60% of the surface of the reaction chamber is patterned with the immobilizing agent.

According to a particular embodiment, at least 70% of the surface of the reaction chamber is patterned with the immobilizing agent.

According to a particular embodiment, at least 80% of the surface of the reaction chamber is patterned with the immobilizing agent.

According to a particular embodiment, at least 90% of the surface of the reaction chamber is patterned with the immobilizing agent.

As mentioned, in one embodiment at least one of the proteins or RNA molecules encoded by the nucleic acid molecules comprises an affinity tag.

In other embodiments, the antibody is specific to one of the proteins, rendering the tag redundant.

An affinity tag is a sequence that generally permits the expressed protein or RNA to be attached to an affinity tag pair with a known orientation. Several different kinds of affinity tags are known in the art. In particular embodiments, the affinity tag is selected from the group consisting of hemagglutinin (HA), AviTag™, V5, Myc, T7, FLAG, HSV, VSV-G, His, biotin, or streptavidin.

The phrase “affinity tag pair” refers to an agent that binds specifically to the affinity tag. In one embodiment, the affinity tag pair serves as the binding agent. The affinity tag pair is typically immobilized on the surface of the chamber.

According to a particular embodiment, the affinity tag is HA and the affinity tag pair is an anti-HA antibody. Preferably, the anti-HA antibodies comprise an affinity tag (e.g. biotin) so that they can be immobilized on the surface of the chamber (or a portion thereof).

Additional agents may be immobilized on to the surface of the chamber to aid in analyzing the association of the components of the complex. In one embodiment, an agent which binds the proteinaceous complex with at least ten fold higher affinity when it is in an assembled form over a non-assembled form is attached to the surface. In the case where the complex is a ribosomal subunit, for example, the present inventors contemplate attaching the second ribosomal subunit to the surface. Binding of the expressed first ribosomal subunit complex to the second immobilized ribosomal subunit complex could serve as a test to analyze correct association of the complex.

Systems which comprise the device of the present invention may comprise a variety of different detection modalities at essentially any location on the microfluidic device. Detection can be achieved using detectors that are incorporated into the device or that are separate from the device but aligned with the region of the device to be detected.

A number of different detection strategies can be utilized with the microfluidic devices that are provided herein. Selection of the appropriate system is informed in part on the type of detectable moiety present in the expressed polypeptide. The detectors can be designed to detect a number of different signal types including, but not limited to, signals from fluorophores, chromophores, polypeptides that emit chemiluminescence, electrochemically active polypeptides, enzymes, cofactors, enzymes and enzyme substrates.

Illustrative detection methodologies suitable for use with the present microfluidic devices include, but are not limited to, light scattering, multichannel fluorescence detection, UV and visible wavelength absorption, luminescence, differential reflectivity, and confocal laser scanning. Additional detection methods that can be used in certain application include scintillation proximity assay techniques, radiochemical detection, fluorescence polarization, fluorescence correlation spectroscopy (FCS), time-resolved energy transfer (TRET), fluorescence resonance energy transfer (FRET) and variations such as bioluminescence resonance energy transfer (BRET). Additional detection options include electrical resistance, resistivity, impedance, and voltage sensing.

Detection occurs at a “detection section,” or “detection region”, namely at the reaction unit where the polypeptide is being expressed. The detection section can be in communication with one or more microscopes, diodes, light stimulating devices (e.g., lasers), photomultiplier tubes, processors and combinations of the foregoing, which cooperate to detect a signal associated with a particular event and/or agent. Often the signal being detected is an optical signal that is detected in the detection section by an optical detector. The optical detector can include one or more photodiodes (e.g., avalanche photodiodes), a fiber-optic light guide leading, for example, to a photomultiplier tube, a microscope, and/or a video camera (e.g., a CCD camera).

Detectors can be microfabricated within the microfluidic device, or can be a separate element. If the detector exists as a separate element and the microfluidic device includes a plurality of detection sections, detection can occur within a single detection section at any given moment. Alternatively, scanning systems can be used. For instance, certain automated systems scan the light source relative to the microfluidic device; other systems scan the emitted light over a detector, or include a multichannel detector. As a specific illustrative example, the microfluidic device can be attached to a translatable stage and scanned under a microscope objective. A signal so acquired is then routed to a processor for signal interpretation and processing. Arrays of photomultiplier tubes can also be utilized. Additionally, optical systems that have the capability of collecting signals from all the different detection sections simultaneously while determining the signal from each section can be utilized.

The detector can include a light source for stimulating a reporter that generates a detectable signal. The type of light source utilized depends in part on the nature of the reporter being activated. Suitable light sources include, but are not limited to, lasers, laser diodes and high intensity lamps. If a laser is utilized, the laser can be utilized to scan across a set of detection sections or a single detection section. Laser diodes can be microfabricated into the microfluidic device itself. Alternatively, laser diodes can be fabricated into another device that is placed adjacent to the microfluidic device being utilized to conduct a thermal cycling reaction such that the laser light from the diode is directed into the detection section.

Detection can involve a number of non-optical approaches as well. For example, the detector can also include, for example, a temperature sensor, a conductivity sensor, a potentiometric sensor (e.g., pH electrode) and/or an amperometric sensor (e.g., to monitor oxidation and reduction reactions). A number of commercially-available external detectors can be utilized. Many of these are fluorescent detectors because of the ease in preparing fluorescently labeled reagents. Specific examples of detectors that are available include, but are not limited to, Applied Precision ArrayWoRx (Applied Precision, Issaquah, Wash.)).

The microfluidic device described herein can be used as a device for analyzing expression of proteins.

Thus, according to another aspect of the present invention there is provided a method of expressing a polypeptide comprising contacting the isolated nucleic acid of the microfluidic device described herein with a composition which comprises enzymes for performing expression of the polypeptide from the isolated nucleic acid, under conditions that allow expression of the polypeptide, thereby expressing the polypeptide.

Selection of the design of a particular microfluidic device for expressing a polypeptide is dependent upon the regulatory elements in the isolated nucleic acid attached to the substrate. The present inventors have shown that gene expression dynamics (e.g. time scale and protein levels) are controlled by the geometrical arrangement of compartments and channels in the microfluidic device. For example the present inventors have shown that the oscillation period of a genetic network is controlled by the channel length.

The fluid which comprises enzymes and additional components for performing expression is typically flowed through the flow channel. At first set-up, liquid is flowed through the flow channel and enters the reaction chambers via the openings by capillary motion. Once the reaction chambers are filled with liquid, typically, components of the cell reaction which are flowed through the flow channel enter the feeding capillary via the opening by diffusion only. The enzymes then diffuse through the feeding capillary and reach the test chamber by diffusion.

The fluid which carries/contains the non-immobilized components are typically buffered solutions which are physiologically relevant such that they do not interfere with expression of the components or interaction therebetween.

In one embodiment, the reaction chamber is heated to physiological temperatures (e.g. 37° C.) to promote transcription/translation and/or association of the components.

Minimal enzymes required to achieve expression of proteins include RNA polymerase, ribosome and aminoacyl tRNA synthetase.

The present invention contemplates addition of these individual enzymes to the composition. Alternatively, the present invention considers use of cell extracts which naturally comprise enzymes for performing expression. An advantage of using a cell extract is that it typically comprises many other factors required to bring about expression of polypeptides. Typically, selection of the cell type from which the extract is prepared is dependent upon the source of the nucleic acids. Thus, for example, if a bacterial promoter sequence is included in the isolated nucleic acid, then a bacterial cell extract should be used.

Additional agents that may be added to the composition include for example dNTPs (ATP, GTP, CTP and UTP), tRNA, coenzyme A, NAD, cAMP, folinic acid, spermidine, agents for energy regeneration such as 3-phosphoglyceric acid or ATPase, DTT, amino acids, Mg-glutamate and K-glutamate.

For eukaryotic systems exemplary agents include RNA polymerase (I) and (II), aminoacyl tRNA synthetase, ribosomes, Initiation+elongation+release factors and energy regeneration enzymes.

In addition, agents may be added to the composition which inhibit the degradation of linear DNA such as the protein GamS.

Additional agents may be selected according to the nucleic acid sequence attached to the substrate. For example, in one embodiment arabinose is added in order to activate an AraC protein dimer.

An exemplary method of preparing a liquid composition that may be flowed through the device according to this aspect of the present invention is described herein below. The liquid part of the cell (cytoplasm) is extracted by breaking the cells. Membranes and insoluble debris are removed by centrifugation. During extract preparation, the endogenous DNA and mRNA may be removed. The extract may be filtered using a 10 kDa molecular weight cut-off filter. Final protein concentration is typically in the order of 1-100 mg/ml, more preferably 1-20 mg/ml, for example 10 mg/ml.

The device of the present invention enables gene expression from the DNA to continue indefinitely, without replacing the genetic material. Nutrients and enzymes are replenished through diffusion. Thus, the expressing may be effected for 2 hours, 4 hours, 6 hours 8 hours, 12 hours, 24 hours or longer.

Long-term expression of proteins allows proteins to reach steady state levels by continuous synthesis and continuous dilution (by diffusion of synthesized proteins through the microchannels and to the flow channel). The device allows visualization in real-time (during expression), binding of in-situ synthesized regulatory proteins to the DNA.

The present invention contemplates a myriad of application for the device described herein, some of which are detailed herein below.
1. A biochip platform for research and development in areas such as systems and synthetic biology, biomedical diagnostics, high-throughput screening, protein expression system;
2. Biological assays in the context of gene expression in spatially defined on-chip reactor systems;
3. A biochip reactor platform for large-scale biosynthesis of molecules (proteins, RNA, peptides, hormones, etc.) with medical applications (e.g. Insulin) based on enzymatic reactions which are currently carried out in bacteria/plants;
4. A platform for embedding schemes of molecular computation in spatially arranged reactors on the chip; and
5. High-throughput analysis of protein functionality resulting from genetic mutations/variations. For example, after mutations/variations in a human genome have been detected, our chip could analyze whether this mutations lead to functionality loss of the expressed protein.

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Materials and Methods

### Fabrication of Microfluidic Chip

Design of the compartment layer, flow layer, and holes location was carried out using Autodesk AutoCAD software. The chip was fabricated on a 4 inch silicon wafer, N-doped <100>, double side polished, and 300 μm thick. The fabrication process consist of the following steps:
1. Compartments layer (reaction unit)
   a. S1818 photoresist spin coated at 2000 rpm for 30 seconds, soft bake at 100° C. for 90 seconds.
   b. UV photolithography using MicroWriter (DMO ML3), dose of 200 mJ/cm²
   c. Developed in Microposit MF-319 (Shipley, Marlborough, MA) for 90 seconds.
   d. Deep reactive ion etching in STSS-ICP to depth of 2.5-3 μm (no Bosch process)
   e. Sonication in acetone for 10 minutes, to remove photoresist.
   f. De-scum in plasma asher, O2 at 5 sccm for 5 minutes to clean wafer.
2. Flow-through layer
   a. S1818 photoresist spin coated at 2000 rpm for 30 seconds on the opposite side of the compartments layer, soft bake at 100° C. for 90 seconds.
   b. UV photolithography using Mask Aligner MA6 and chrome mask (mirrored) in back side alignment mode, exposure for 40 seconds at ˜11 mW/cm²
   c. Developed in Microposit MF-319 (Shipley, Marlborough, MA) for 2 minutes.
   d. Deep reactive ion etching in STSS-ICP to depth of 120-150 μm using Bosch process of 250 cycles.
   e. Sonication in acetone for 10 minutes, to remove photoresist.
   f. De-scum in plasma asher, O2 at 5 sccm for 5 minutes to clean wafer.
3. Through-holes (openings)
   a. AZ4562 photoresist spin coated at 2000 rpm for 30 seconds over the compartment layer, soft bake at 100° C. for 90 seconds.
   b. UV photolithography using MicroWriter (DMO ML3), dose of 650 mJ/cm²
   c. Develop in AZ351-B developer for 2 minutes.
   d. Coat another 4 inch wafer with thermal grease (heat to 70° C.), and stick the fabricated wafer with the flow layer facing the grease. This prevents the wafer from breaking during the etching process and provides thermal contact to the cooling mechanism in the ICP machine.
   e. Deep reactive ion etching in STSS-ICP Bosch process of 600 cycles, to achieve depth of ˜200 μm for the small features.
   f. Heat the wafers to 70° C. and detach the fabricated chips.
   g. Put the chips in a chips holder and sonication in acetone for 20 minutes, to remove photoresist.
   h. De-scum in plasma asher, O2 at 5 sccm for 10 minutes to clean chips.
4. SiO₂ coating
   a. The fabricated chips are coated with a 50 nm SiO₂ layer in both sides, using plasma enhanced chemical vapor deposition (Plasma-Therm VERSALINE, Saint Petersburg, Florida, USA). This enables DNA brush assembly (as described below), and increases adhesion forces with PDMS.

### DNA Brush Assembly:

To enable patterning of DNA brushes on the SiO₂ coated wafer, a biocompatible photo-cleavable monolayer was deposited on the wafer. The monolayer is a polymer composed of a polyethylene glycol backbone with a Nvoc-protected amine at one end, and a trialkoxysilane function at the other end. The polymer was dissolved in Toluene with concentration 0.2 mg/mL, and incubated on the surface of the wafer for a duration of 20 minutes, for the monolayer to form. After the incubation, the wafer was washed in Toluene and dried.

The reactive amine groups were exposed to UV light using MicroWriter (DMO ML3) dose of 1000 mJ/cm². Biotin N-hydroxysuccinimidylester (biotin-NHS) dissolved in a borate buffered saline (0.5 mg/ml) was incubated on the wafer for 30 minutes. The biotin covalently bonded with the UV exposed amine groups on the monolayer, attaining a surface pattern. Linear double stranded DNA fragments were made by polymerase chain reaction (PCR) with KAPA HiFi HotStart ReadyMix (KAPA BIOSYSTEMS), using one primer with biotin and another with Alexa Fluor-647 fluorophore, both attached at the 5′-end (IDT).

The final DNA solution contained Streptavidin (SA) conjugated to DNA at a concentration of 100-300 nM in a phosphate buffered saline (PBS) and a 7% glycerol for droplet stability. The locations of the different DNA constructs to be placed on the chip were generated automatically using a custom software. The generated XML file was loaded to the GIX Microplotter II (Sonoplot Inc., Middleton, WI), and aligned with the physical chip. The spotter then automatically executed deposition of nano-liter DNA-SA droplets onto the compartments using the under controlled humidity of 50-60%, and left to incubate for at least 2 hours (can also be left overnight).

### Cell-Free Protein Synthesis (CFPS) Preparation

An E. coli cell-free transcription-translation (TX-TL) reaction was carried out as described in Garamella et al., ACS Synth. Biol., vol. 5, no. 4, pp. 344-355, 2016. The cell extract provided the necessary machineries for gene expression. The cell extract contained active proteases and ribonucleases. The endogenous E. coli RNA polymerase, allowed for the use of the repertoire of the E. coli regulation toolbox, drove the transcription process. T7 polymerase was added at a concentration of 150 nM to the reaction mix to enable constant activity from the P_{T7} promoter. Proteins with degradation tags, such as ssrA, were targeted to the ClpXP degradation complex.

The protein GamS from lambda phage, was added in concentration of 3 μM to inhibit the degradation of linear DNA by the 3′ exonuclease activity of the RecBCD complex which was present in the cell-free system.

### Experiment Setup and Workflow

Preparation of the device was conducted as follows:
1. The spotted chip was washed in PBS to remove non-specific DNA, and thoroughly dried using N2 gun.
2. The compartment layer was sealed by placing PDMS covered coverslip on top of it, and gently pressed to remove trapped air.
3. The chip was flipped and the flow layer was sealed using a thick PDMS slab, with two holes punched in it, for the inlet and outlet tubing.
4. The chip was placed in a custom made aluminum holder, designed to exert uniform pressure on both sealing layers.
5. The assembled device was inserted to a temperature controlled incubator at a temperature of 31° C., on the microscope's translation stage.
6. PBS was flown through the flow channels by gentle manual pulling on a syringe. The device was kept in PBS for ˜30 minutes to allow the liquid to diffuse and fill all compartments.
7. The syringe was placed in a syringe pump (PHD ULTRA, Harvard Apparatus), and cell extract, incubated in 4° C. using a thermoelectric cooler, was flown to replace the PBS in the chip. The initial flow rate is ˜5 μL/min, to allow homogeneous initial conditions across the chip. After the PBS was replaced (few minutes, depending on the length of the inlet tube), the pump rate was switched to the desired rate of 0.3 μL/min.

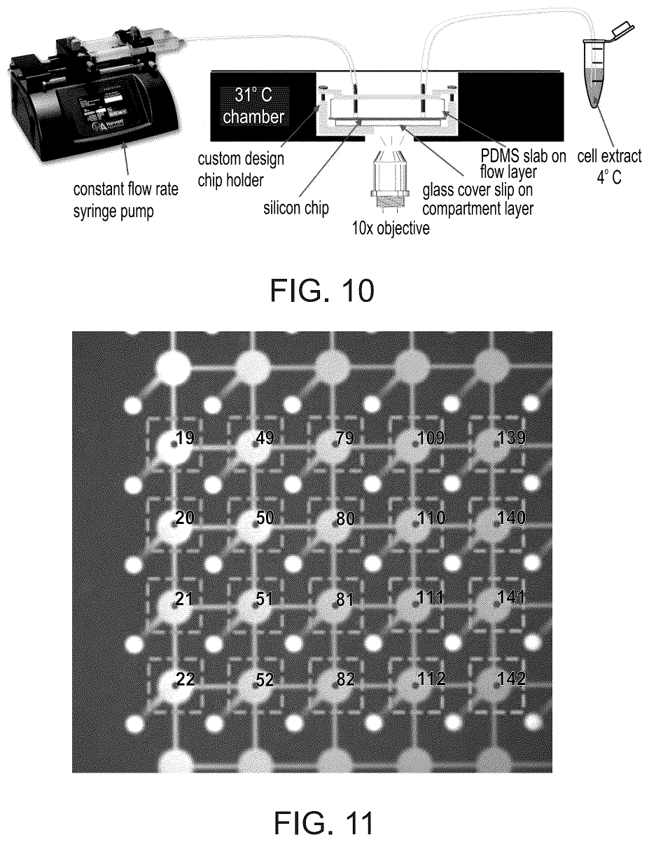

An example of the experimental setup is shown in FIG. 10.

### Imaging

The experiment was conducted on a translational stage coupled to an inverted microscope (Zeiss Axiovert 200M) with ANDOR Neo sCMOS camera (Andor Technology, Belfast, UK) and X10 Zeiss UPLFLN U plan objective. The Semrock filters set was used: for 647 nm red DNA fluorophore marker, and for 488 nm eGFP detection. Exposure times varied between 300-1000 ms depending on the experiment, and the time interval between consecutive images was 2 minutes. The light source was X-cite120Q.

### Image Processing and Analysis

Data extraction from the images was automated using proprietary software. First, the images produced by the microscope were aligned to the original design using the location of two position markers, etched on the chip. This allows to compute the transformation matrix (rotation and scale), between the design coordinates, and the microscopes coordinates. Next, the program identified the location of each compartment in the images, and cut a square centered at each compartment with face length 150% of the compartment diameter (to overcome minor misalignment), as shown in Error! Reference source not found. The background level was estimated by computing the 7 percentile of the extracted squares, and the total signal was estimated as the 90 percentile, for each time point. Finally, the dynamics in each compartment was obtained by subtracting the background level from the total signal.

Oscillation period was computed by calculating the Fourier transform of the signal, and finding the peak frequency in the range corresponding to period of 1-5 hours. When analyzing collective oscillator dynamics, local maxima of the signal were found with sufficient prominence, to get the oscillation peak times.

### Results

### Chip Architecture and Design

### Feeding Configuration

To allow a 2D networks of interconnected compartments while keeping each compartment in steady-state, a third dimension must be exploited. The purpose of the feeding capillary is to continuously dilute out the synthesized proteins by diffusion and replace the cell-free extract that provides the energy and machinery for gene expression. Therefore the feeding capillaries must connect to the main flow of cell extract independently of the coupled network topology. FIG. 1A shows a general scheme of the biochip designed to allow long-term gene expression in coupled 2D networks. The DNA compartments, connecting and feeding capillaries were fabricated on one face of a 300 μm thick silicon wafer, while the flow-through channel is etched off the opposite face to a depth of 150 μm. Circular through-holes (openings) of diameter 30 μm are etched to connect the compartments and the flow-through channel, which implies that the depth of the hole is approximately 150 μm. SEM image of through hole is shown in FIG. 1D. To maintain a diffusive regime in the compartment layer and counteract the effect of the flow of the cell extract in the flow channel, a high hydrodynamic resistance is created in the capillary and DNA chamber by carving them to a shallow depth of 2.5 μm.

### DNA Deposition

Spotting nanoliter droplets of DNA on the chip enables embedding synthetic genetic programs with high spatial accuracy at each compartment of the network.

### Flow Layer Design

The design of the flow layer has to achieve the following goals:
1. Minimize the induced flow at the compartments layer to maintain diffusive regime
2. Maintain homogeneous velocity profile across the chip to make compartment feeding independent of the position in the network
3. Filter the incoming cell extract and minimize air bubble formation to prevent blockage of capillaries

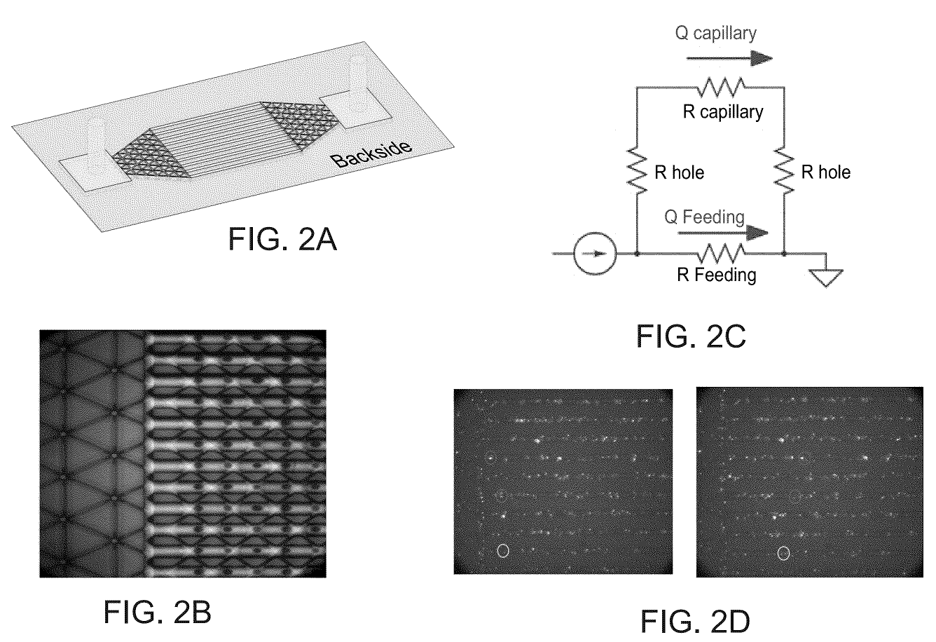

FIG. 2A shows a scheme of the flow layer developed for this example. The compartments layer is fabricated on the opposite face of the silicon wafer, and aligned such that the edge of the feeding capillaries sit directly above the horizontal flow channels, so the through-holes directly connect the layers. FIG. 2B shows the horizontal flow channels (the black circles are the holes). In order to analyze the flow in the device, we start by estimating the Reynolds number

$${R}_{e} = \frac{vL}{v} ,$$

where L is the characteristic linear dimension, v is the flow speed, and v is the kinematic viscosity of the fluid. Taking typical flow rate and dimensions of the chip we estimate Rₑ≈0.04<<1, so we can assume that all flow is laminar, which implies linear relation between pressure difference and flow rate (i.e. can be treated as a DC circuit). The flow rate in the compartment layer (through the feeding capillaries) can be estimated from solving the electric analogue shown in FIG. 2C. The through hole is a cylinder, thus it's hydrodynamic resistance is

$${R}_{hole} ∝ \frac{8 \cdot L}{π {R}^{4}}$$

The resistance of the feeding capillary has rectangle cross section, thus it's hydrodynamic resistance is given by

$${R}_{capilary} ∝ \frac{12 \cdot l}{w \cdot {h}^{3}}$$

For typical dimensions used in our design (L≈150 μm, R≈15 μm, l≈100 μm, w≈5 μm, h≈3 μm), we get

$$\frac{{R}_{capillary}}{{R}_{hole}} \approx 40 ≫ 1$$

so the resistivity of the hole can be neglected.
The residual flow rate in the capillary is calculated by imposing equal pressure difference on both branches: ΔP=Q_{feed}R_{feed}=Q_{cap}R_{cap} where from conservation of fluid Qₜₒₜₐₗ=Q_{feed}+Q_{cap} so we can substitute and get:

$${Q}_{cap} = {{Q}_{total} ( 1 + \frac{{R}_{feed}}{{R}_{cap}} )}^{- 1}$$

As both the capillary and the flow-through channel have rectangular cross section, the ratio of hydrodynamic resistances is

$$\frac{{R}_{feed}}{{R}_{cap}} = \frac{W \cdot {H}^{3}}{w \cdot {h}^{3}} ≫ 1$$

and we can neglect the 1 in the brackets and get:

$${Q}_{cap} = {Q}_{total} \frac{{R}_{feed}}{{R}_{cap}} = {Q}_{total} \frac{w \cdot {h}^{3}}{W \cdot {H}^{3}}$$

The flow rate manifest in fluid velocity:

$${v}_{cap} = \frac{{Q}_{cap}}{w \cdot h} = {Q}_{total} \frac{{h}^{2}}{W \cdot {H}^{3}}$$

Finally, the criterion for diffusive regime is equivalent to demanding that the time it takes a protein to diffuse to a neighboring compartment is much shorter than the time it would have taken the protein to be transported there due to the residual flow:

$${t}_{drift} = \frac{l}{{v}_{cap}} ≫ {t}_{diff} = \frac{{l}^{2}}{D} \to {v}_{cap} ≪ \frac{D}{l} \approx 0.5 \frac{μ m}{s}$$

Where we used a diffusion coefficient for a typical protein as

$$D \approx 50 \frac{μ {m}^{2}}{s}$$

To accommodate this demand, we use a parallel flow channel layer containing 30 channels with cross section dimensions H≈100 μm, W≈150 μm, and syringe pump with rate

$$0.3 \frac{μL}{min} .$$

Then the drift velocity

$${ν}_{cap} \approx \frac{0.3}{30} \cdot \frac{{3}^{2}}{150 \cdot 10 {0}^{3}} \approx 1 {0}^{- 2} \frac{μm}{s}$$

which indeed satisfies the diffusive criterion.
Recall that we also need to maintain rapid flow in the feeding channel in order to supply the reactors with fresh cell extract and uphold the ZBC for the produced protein.
The flow velocity in the feeding channel

$${ν}_{feed} \approx \frac{{Q}_{total}}{W \cdot H} \approx 10 \frac{μm}{s} ,$$

creating ZBC scenario for the proteins synthesized in the compartments.

This architecture also satisfies the other two design considerations: The network of capillaries (20 μm) serve both as a filter for the incoming extract, and creates the conditions for homogeneous velocity profile in the flow-through channels because the shortest path leading from the inlet is the same for all channels. Note that the edge channels tend to maintain lower flow rate (hence lower velocity) because there are less paths through the filter network that lead to them, so the hydrodynamic resistance from the inlet is higher.

To minimize air bubble formation in the initial stage of filling up the sealed chip with PBS, the flow-through channels are narrow (100 μm) which creates higher flow velocity and pressure gradient. Thin capillaries were also added between the flow channels, so in case a bubble is formed it will not block the entire channel (causing shut-down of ˜30 compartments) and the flow will bypass it (so only 1-2 compartments will be affected).

### Single Compartment Theory-Diffusive Model

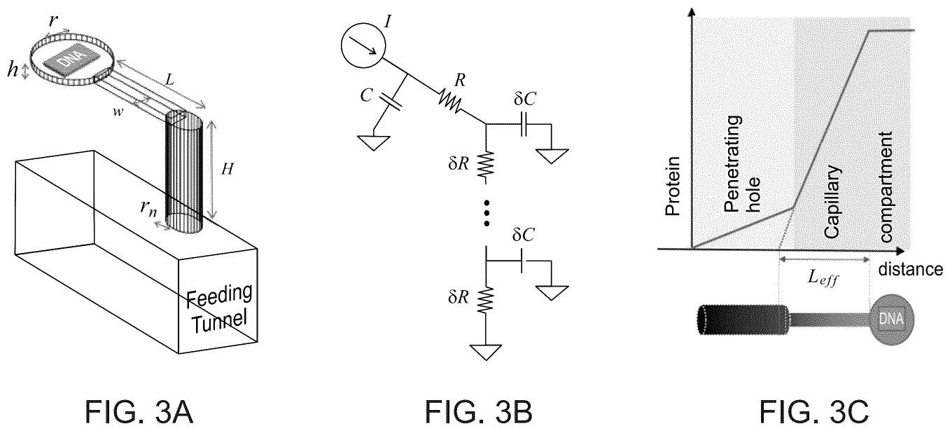

In an exemplary chip, a compartment is connected to the flow-through channel by etching vertical through-hole in the silicon wafer, thus shifting the zero boundary condition (ZBC) to the hole-flow channel junction. In a typical chip, the volume of the hole is roughly 10 time the volume of the compartment, but its length is comparable to the capillary (up to 2-fold). To model this, we use a diffusive electrical analogue approach known as Transmission Line Matrix (TLM) (11). The protein concentration is represented as the voltage V, the physical cavities of the device are discretized and modeled by connected capacitors and resistors in an array (FIG. 3A), where the “capacitance” C is the volume of the discretized unit, and the “resistance” is its length divided by the area of contact to the adjacent unit. We show that a 1D-discrete diffusion equation arise from solving this electrical model.

As the contact area of compartment with the capillary is small we can neglect its “resistance” and model it as a capacitor, and we can also neglect the “capacitance” of the capillary as its volume is much smaller than the compartment. The through hole is discretized to roughly 10 identical units of length δx, such that each unit's “capacitance” is the order of the compartment. The resulting electrical analogue of the compartment-capillary-hole is shown in FIG. 3B. The dependence of the electrical parameters on the physical parameter is summarized in the Table 1 below.

TABLE 1

| Element in | Physical | | Electrical | |
|---|---|---|---|---|
| the chip | representation | Model | analogue | Comments |
| Protein | Fluorescent | voltage | V(x) | |
| concentration | signal | | | |
| DNA brush | | current | I(x = 0) | Function of |
| as protein | | source | | regulation, |
| source | | | | extract freshness, |
| | | | | total DNA |
| | | | | amount etc. |
| Compartment | r-radius | capacitor | C = πhr² | Internal |
| | h-depth | | | resistance |
| | | | | neglected |
| Capillary | L-length w-width | resistor | $R = \frac{L}{w h} \cdot {D}^{- 1}$ | Internal capacity neglected, |
| | h-depth | | | diffusion |
| | | | | coefficient was |
| | | | | arbitrarily added |
| Hole | H-length rₕ -radius | discrete cascade of | $δ R = \frac{δ H}{π {r}_{h}^{2}} {D}^{- 1}$ | δH is such that δC is in |
| | | resistors | $δ C = π {r}_{h}^{2} δ H$ | the order of the |
| | | and | | capacitance of |
| | | capacitors | | the compartment. |

Using Milman's theorem for the circuit in Error! Reference source not found. 3B, and using the formula for the impedance of a capacitor

$${Z}_{c} = \frac{1}{j ω C} ,$$

we can develop an equation for the “voltage” in the hole:

$$V ( x ) = \frac{\frac{V ( x - δ x )}{δR} + \frac{V ( x + δ x )}{δ R}}{\frac{1}{δ R} + \frac{1}{δ R} + j ωδ C} ⇒ j ω V ( x ) δ R δ C = V ( x + δ x ) + V ( x - δ x ) - 2 V ( x )$$

Plugging the expressions for δR and δC, we get:

$$j ω V ( x ) = D \frac{V ( x + δ x ) + V ( x - δ x ) - 2 V ( x )}{δ {x}^{2}}$$

To transform this equation from the frequency (Fourier) domain to the time domain, we replace

$$\to \frac{\partial }{{\partial }_{t}} ,$$

and get a discretized 1D diffusion equation:

$$\frac{\partial V ( x )}{\partial t} = D \frac{V ( x + δ x ) + V ( x - δ x ) - 2 V ( x )}{δ {x}^{2}}$$

Note that in the short hole limit H→0, this model reduces to a single capacitor and resistor with a current source (RC circuit):

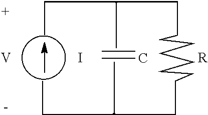

The voltage (protein concentration in the compartment) is given by:

$$V = I \frac{R}{1 + j ω RC}$$

Transforming this equation to the time domain gives the following first order ODE:

$$\frac{\partial V}{\partial t} = \frac{I}{C} - \frac{V}{RC}$$

For the steady state analysis, we treat the capacitors as open circuit and we can sum the resistivities:

$${R}_{tot} = {R}_{cap} + {R}_{hole} = \frac{L}{w h D} + \frac{H}{π {r}_{h}^{2} D} = \frac{L}{w h D} ( 1 + \frac{w h H}{π {r}_{h}^{2} L} ) {=}^{Δ} \frac{{L}_{eff}}{w h D}$$

This form suggests the through-hole effectively elongates the capillary by a relative factor:

$${L}_{eff} = L + Δ L , \frac{Δ L}{L} = \frac{w h H}{π {r}_{h}^{2} L}$$

The resulting protein concentration is visualized in Error! Reference source not found. 3C. The effective elongation is marked in red, where the effective ZBC occur.

For a typical chip configuration, this elongation corresponds to a 10% elongation, which means that the effective ZBC occur close to the capillary-hole junction, thus not imposing significant constraint on the minimal lifetime of a compartment in the chip architecture.

### Validation of Hole Model

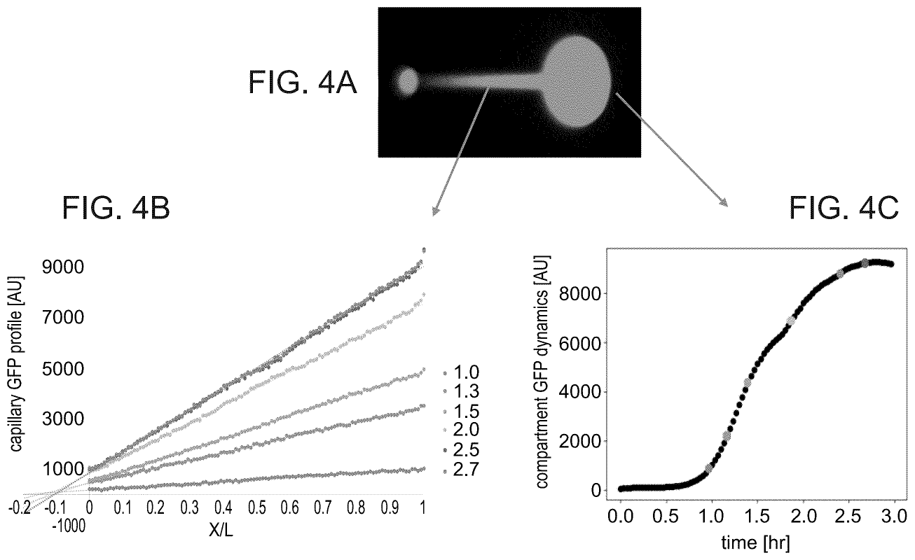

The fundamental test for the presently disclosed chip design is validating the diffusive model described above. The steady state solution of the 1D-diffusion equation subject to two different concentration at the boundaries is a linear gradient (constant slope). We expressed GFP under a strong promoter (σ70) without any regulation, FIG. 4A shows a compartment and the profile of GFP concentration along the capillary at different time points and expression values. Linear gradient is evident, suggesting that synthesized GFP in the reactor is diluted out through the capillary by diffusion.

In the current chip configuration the length of the capillary is roughly twice shorter than the length of the hole Hₕₒₗₑ≈2L=150 μm, thus if the ZBC would naively occur at the hole-flow channel junction, the concentration at the capillary-hole junction would be ⅓ of the compartment concentration. FIG. 4C shows that the concentration at the capillary-hole junction (x=0) is not zero, but much less than ⅓ of the compartment concentration, meaning the ZBC occur close to the edge of the feeding capillary.

To find the shift of the ZBC from the capillary-hole junction, we fit each profile to a linear line and extrapolated it into the through-hole until zero concentration is reached (intersection with the x-axis). FIG. 4B shows that all profiles intersect roughly at the same point, which is a good estimate for the location of the effective ZBC, because by definition it is the location where the protein concentration is zero. The extrapolated value from the experiment is ˜0.1L, which is compatible with the prediction of the diffusive model, for the physical dimensions of the cavities.

This shows that the exemplary chip architecture does not impose a large inherent minimal capillary length due to the existence of the hole, which linearly affects the minimal possible lifetime. Avoiding this scenario is very important, so as to work with short lifetimes in order to achieve high oscillation frequencies.

### Lifetime Parameters Scan of Unregulated Expression

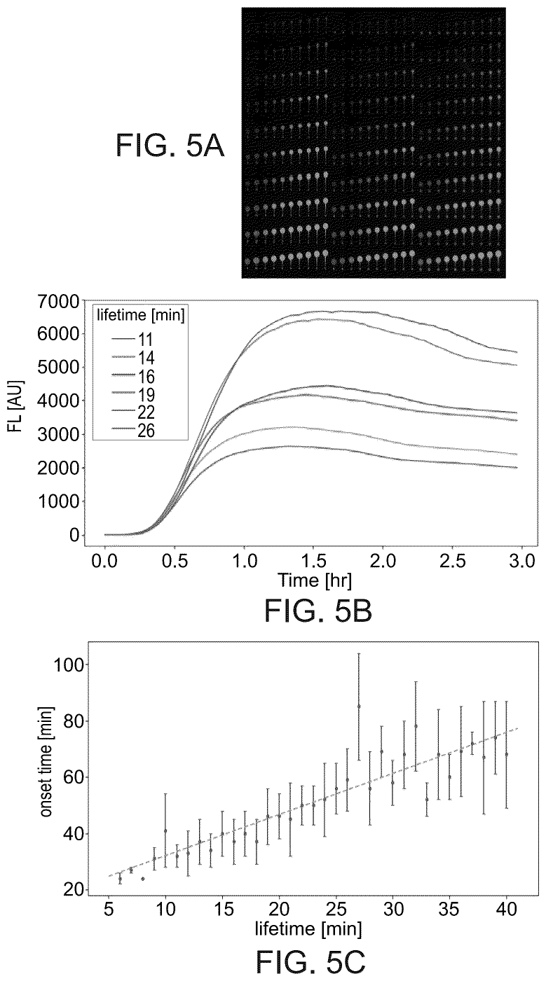

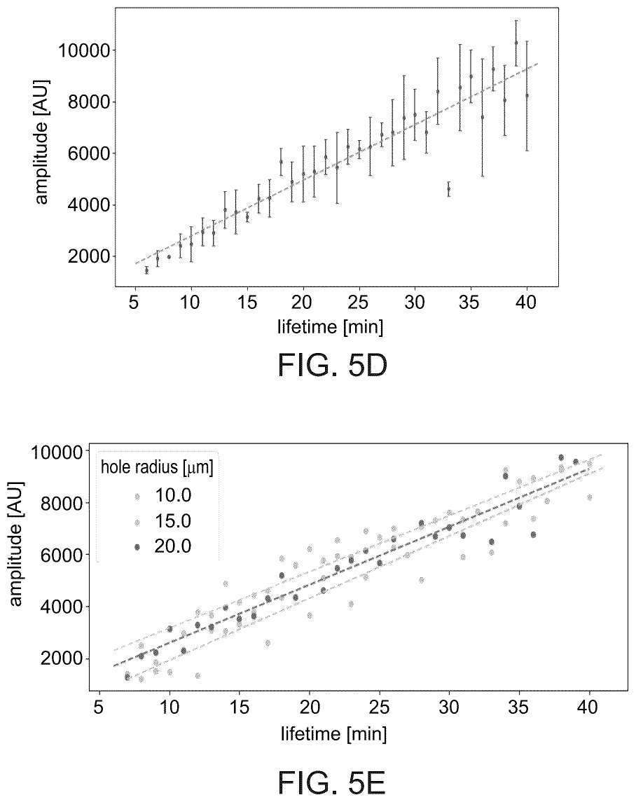

Next a chip consisting of 300 uncoupled compartments was fabricated, varying the R and L parameters (10 values for each parameter, and 3 replicas of each compartment with varying hole radius), as shown in FIG. 5A.

The area patterned with DNA was kept the same for all compartments radii (such that the expression rate will be influenced only by the geometry).

In order to estimate the time to reach steady state, we need to take into account the time it takes for the cell extract to diffuse along the capillary and reach the compartment to initiate gene expression:

$${T}_{on} = {T}_{peak} - {T}_{diff} = {T}_{peak} - \frac{{L}^{2}}{D}$$

FIG. 5C shows linear dependence between Tₒₙ (averaged over all compartments with similar lifetime, quantize to 1 min) and the lifetime of the compartment, as predicted by the model.

FIG. 5D shows the average steady state amplitude for different lifetimes. A good linear fit is evident. In FIG. 5E, we differentiate between the three replicas of compartments by the hole radius. Linear relation between steady state and lifetime is kept, and no significant effect of the radius is observed, further validating the diffusive model.

### Genetic Oscillator in the 2D Chip

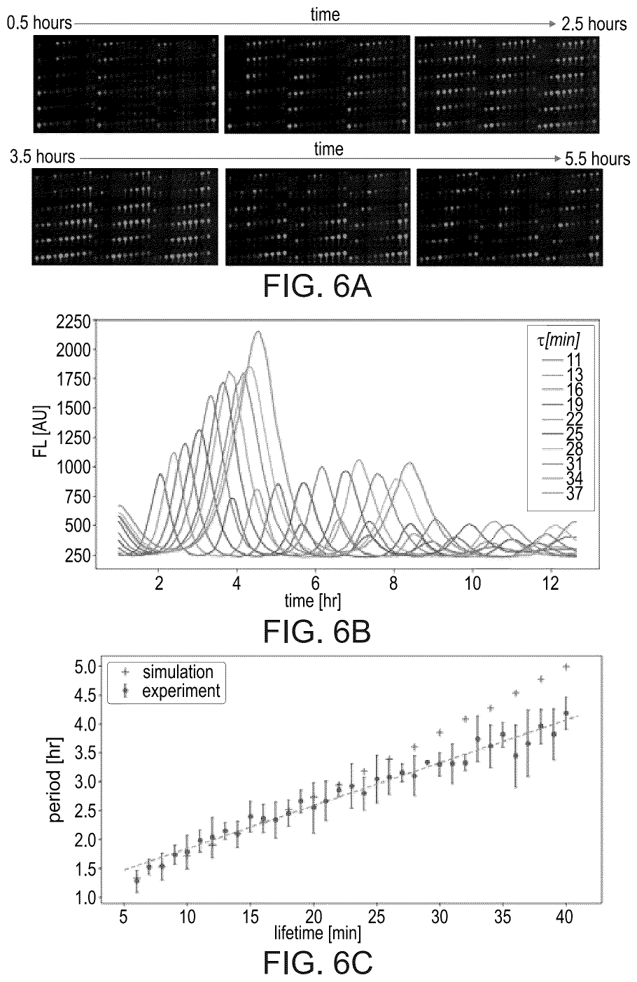

Next, a genetic oscillator gene construct was introduced to the biochip. FIG. 6A shows gene expression on a chip. The GFP reporter dynamics in the compartments (FIG. 6B) undergoes spontaneous oscillations with varying oscillation period (frequency). FIG. 6C shows clear linear relation between the geometrical lifetime, for different combinations of compartment dimension, which is predicted by numerical solution of the oscillator equations. Note that the slope between the measurements and the simulation is different, which can be fine-tuned by better estimating the bio-chemical parameters in the oscillator equations.

### Localized Perturbation:

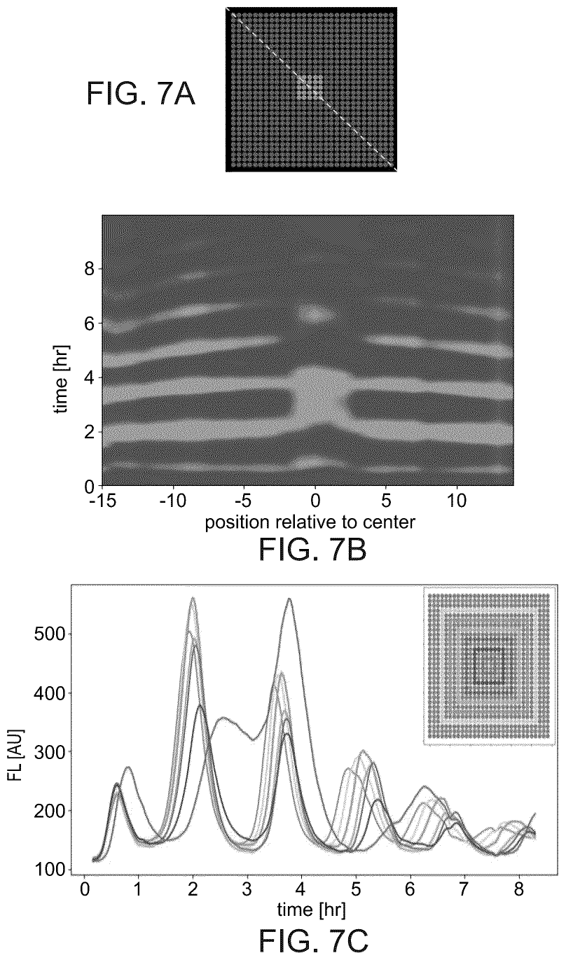

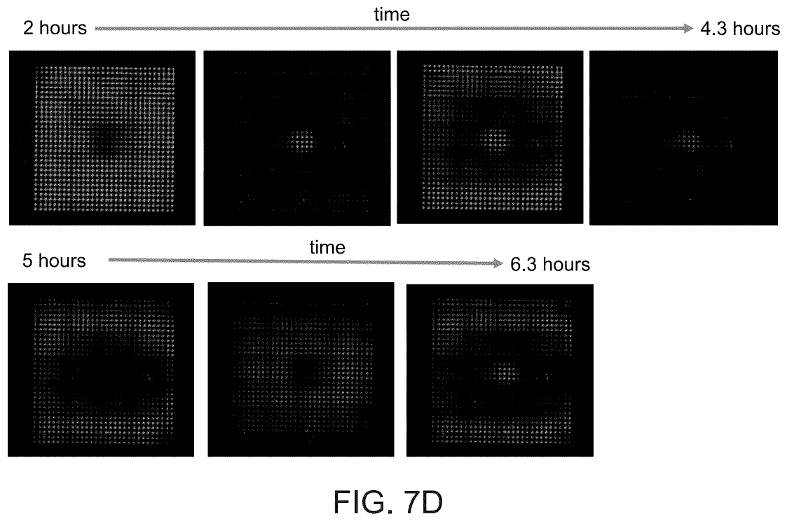

The simplest form of symmetry breaking that can be introduced into the array is adding local population of intrinsically slower oscillator in the center of the array, populated with identical oscillators. To this end, a chip was fabricated with a square lattice of 30×30 compartments, and altered the geometry of 25 compartments located at the center or the array, such that their natural oscillation period is longer than the rest (FIG. 7A). This design does not break angular symmetry and thus compatible with the external boundary effect observed previously, but alters the local interaction between compartments near the center of the array.

We observed no significant effect on the collective behavior at scale of the array, i.e. the propagation of the boundary conditions inward and the intrinsically slower period of the center compartments, except a modification of the oscillatory dynamics in the slower population and in the adjacent compartments. These compartments exhibited a change in frequency, manifested in merging of the second and third peaks, as shown in FIG. 7C.

### Pseudo 1D Perturbation

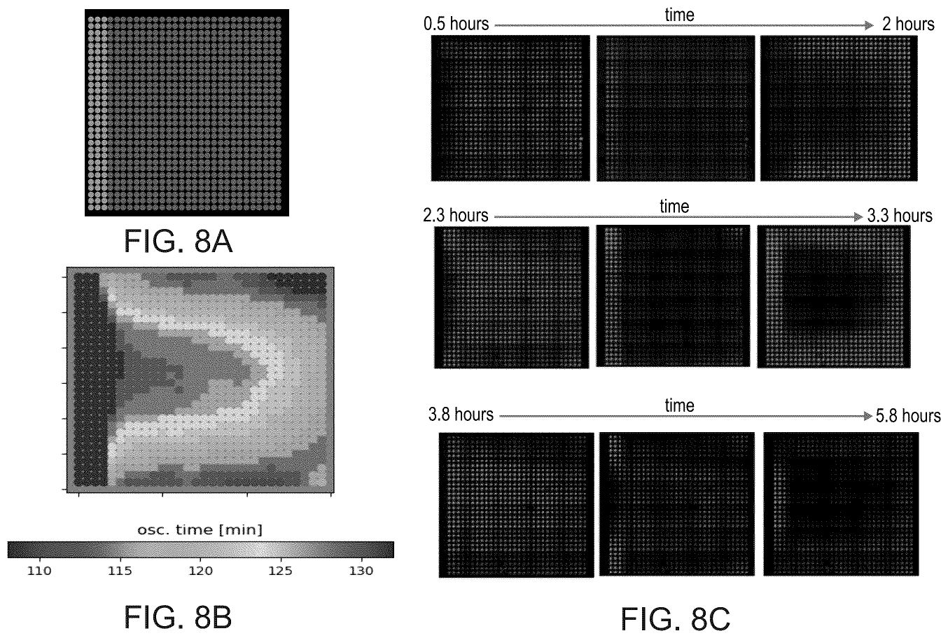

In this configuration, the slower oscillators population was patterned in the left 3 columns of the square array. This design breaks the symmetry only the x-direction, so in the absence of boundary effects discussed in previous chapter, we expect this chip behave collectively like a 1D array. FIGS. 8A-C show that the effect of the symmetry breaking combined in a non-linear fashion with the boundary effect creates a richer spatial wave phenomenon. The first oscillation of the two population is to a high degree decoupled and homogeneous for each population. The second oscillation of the fast oscillators starts with the external boundary, but the slow oscillators break the angular symmetry and create a curved collective behavior propagating from the bulk center of the array towards the left side, maintaining symmetry with respect to the x-axis. For the subsequent oscillations, these dynamics repeat, with variations due to difference in oscillation periods between the populations.

FIG. 8B demonstrates the dynamics by displaying the time of the second oscillation for each compartment in the array, and the slow oscillators on one side of the chip break the symmetry of the external boundary effect.

### Interacting Localized Perturbations:

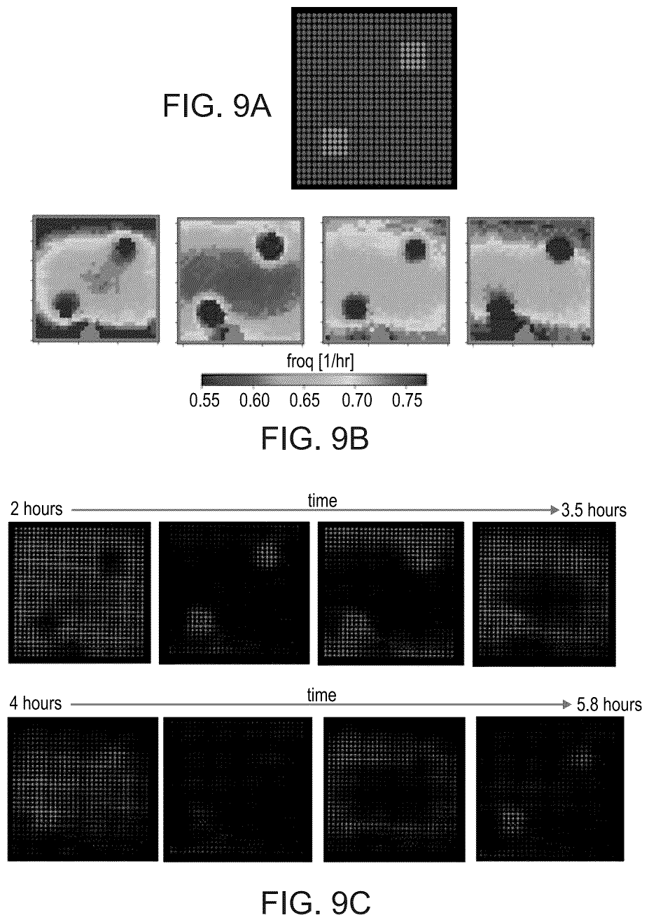

To further examine the effect of localized perturbations in the lattice, and possibly probe long-range interactions manifest between these sources, a chip was fabricated with two localized populations of slow oscillators (25 compartments in each) on the diagonal of the lattice (FIG. 9A). This network topology breaks the angular symmetry of the external boundary, and introduces new symmetry with respect to the opposite diagonal. It also brings the perturbations (sources of slower oscillation) much closer to the array boundary, compared to the topology with the single perturbation in the center. As in the previous network topologies, the first oscillation of each population is homogeneous and independent of the other population. The second oscillation, occurring ˜3 hours into the experiment, shows the effect of the external boundary condition of the array, and the imposed boundary condition by the network structure. FIG. 9B shows the frequency changes in each compartment in the network, for the duration of 4 oscillations, starting from the second. Initially, the system maintains the diagonal symmetry imposed by the internal boundary conditions, and there is entrainment of oscillators located on the diagonal between the slower sources. The modulation of the oscillation in compartments adjacent to the perturbations, described in the central perturbation section, is repeated here in the third oscillation, manifesting in higher frequency comparted to the bulk of the fast oscillators. Consequently, this symmetry is replaced by symmetry with respect to the x-axis (except for the two actual slow populations), possibly as a result of a non-linear interaction between the external and internal boundary conditions.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A microfluidic device comprising:
(i) a first surface which comprises a plurality of reaction units and at least one opening to a hollow, each reaction unit having a test chamber connected to said at least one opening via a feeding capillary, wherein said at least one opening is in communication with said hollow along a depth of the device, said hollow being defined by internal walls; and
(ii) a second surface which comprises at least one flow-through channel having at least one inlet port and at least one outlet port,
wherein said hollow extends to said second surface and fluidly connects said test chamber with said flow-through channel.

2. A microfluidic device comprising:
(i) a first surface which comprises a plurality of reaction units and a plurality of hollows, each reaction unit having a test chamber connected to at least one opening via a feeding capillary, wherein said at least one opening is in communication with a hollow of said plurality of hollows along a depth of the device, said hollow being defined by internal walls, wherein a biomolecule is attached to a surface of at least a first portion of said test chamber, said biomolecule being a component of a cellular process, and wherein an immobilizing agent is optionally attached to a surface of at least a second portion of said test chamber, said immobilizing agent capable of immobilizing a product of said cellular process; and
(ii) a second surface which comprises at least one flow-through channel having at least one inlet port and at least one outlet port,
wherein said hollow extends to said second surface and fluidly connects said test chamber with said flow-through channel;
wherein said at least one flow-through channel, said feeding capillary and said at least one opening being of dimensions, so as to ensure said cellular process reaches and is maintained in a steady-state.

3. The microfluidic device of claim 1, wherein said at least one flow-through channel, said feeding capillary and said at least one opening being of dimensions to allow diffusion of a molecule from said flow-through channel via said hollow to said test chamber of said reaction unit and vice-versa and reduce flow of fluid in said reaction unit.

4. The microfluidic device of claim 2, wherein said at least one flow-through channel, said feeding capillary and said at least one opening being of dimensions to allow diffusion of a component of a cellular process from said flow-through channel to said test chamber of said reaction unit via said hollow and reduce flow of fluid in said reaction unit.

5. The microfluidic device of claim 1, wherein at least one said reaction unit further comprises at least one interconnecting capillary which connects a test chamber of a first reaction unity of said plurality of reaction units to a test chamber of a second reaction unit of said plurality of reaction units.

6. The microfluidic device of claim 1, wherein at least a portion of said plurality of reaction units are organized in a grid such that at least one test chamber of a reaction unit of said plurality of reaction units is connected to four test chambers of corresponding reaction units via interconnecting capillaries.

7. The microfluidic device of claim 1, wherein the depth ratio of said reaction unit: flow-through channel is greater than 1:5.

8. The microfluidic device of claim 1, wherein the hydrodynamic resistance in said feeding capillary is at least 10³ times greater than the hydrodynamic resistance in said flow-through channel.

9-10. (canceled)

11. The microfluidic device of claim 1, wherein the width ratio of said feeding capillary: flow-through channel is greater than 1:3.

12. The microfluidic device of claim 1, wherein the depth of the reaction unit is about 1 micron to about 50 microns and the depth of the flow-through channels is about 20 microns to about 250 microns.

13. (canceled)

14. The microfluidic device of claim 1, wherein the diameter of said hollow is between 5-50 microns.

15. The microfluidic device of claim 1, wherein a volume of said hollow is about 3-10 times a volume of said test chamber.

16-20. (canceled)

21. The microfluidic device of claim 1, wherein a biomolecule is attached to a surface of at least a portion of said test chamber, said biomolecule being:
(i) a component of a cellular process; and/or
(ii) an immobilizing agent capable of immobilizing a product of said cellular process.

22. (canceled)

23. The microfluidic device of claim 21, wherein said component of a cellular process comprises a nucleic acid.

24. The microfluidic device of claim 23, wherein a sequence of said nucleic acid encodes a promoter operatively linked to a nucleic acid sequence encoding a polypeptide.

25-36. (canceled)

37. The microfluidic device of claim 21, wherein said immobilizing agent comprises an antibody.

38. The microfluidic device of claim 21, wherein said product of said cellular process comprises a ribosome.

39. (canceled)

40. A method of expressing a polypeptide comprising contacting the isolated nucleic acid of the microfluidic device of claim 24 with a composition which comprises components for performing expression of the polypeptide from said isolated nucleic acid, under conditions that allow expression of the polypeptide, thereby expressing the polypeptide.

41-42. (canceled)

43. The method of claim 40, wherein the protein forms a gradient in the reaction unit.

44. The method of claim 40, wherein the expressing is effected for at least 2 hours.
